(19) United States
(12) Patent Application Publication
(10) Pub. No.: US 2015/0111259 A1
(43) Pub. Date: Apr. 23, 2015

(54) Method for Making High Maltose Syrup

(71) Applicant: Danisco US Inc., Palo Alto, CA (US)

(72) Inventors: T. Kleinhout, The Hague (NL); Bart Christiaan Koops, Pijnacker (NL); Floor K. Kooy, Leiden (NL); Sung Ho Lee, North Liberty, IA (US); Jayarama K. Shetty, Pleasanton, CA (US); Bruce A. Strohm, Beloit, WI (US)

(73) Assignee: DANISCO US INC., Palo Alto, CA (US)

(21) Appl. No.: 14/387,779

(22) PCT Filed: Mar. 12, 2013
(86) PCT No.: PCT/US2013/030384

§ 371 Date: Sep. 24, 2014

Related U.S. Application Data
(60) Provisional application No. 61/616,990, filed on Mar. 28, 2012.

(51) Int. Cl. C12P 19/12; C12P 19/14

(52) U.S. Cl. CPC C12P 19/12; C12P 19/14
USPC 435/99; 435/201

(57) ABSTRACT

The present teachings provide direct conversion of granular starch into a soluble sugar composition comprising a high, very high, and/or ultra high maltose content. The method involves contacting an aqueous slurry of granular starch with an enzyme composition comprising an appropriate ratio of an alpha-amylase and a maltogenic enzyme to produce a soluble starch substrate that is enzymatically converted to a sugar composition containing higher maltose. The process may use more than one temperature to achieve the same.

### CROSS REFERENCE TO RELATED APPLICATION

This application claims the benefit of U.S. Provisional Application No. 61/616,990, filed 28 Mar. 2012, which is herein incorporated by reference in its entirety.

### FIELD OF THE INVENTION

This disclosure is directed to compositions and methods for using enzymes to make high maltose syrups from starch substrates.

### BACKGROUND OF THE INVENTION

Maltose, a di-saccharide composed of two D-glucopyranoses joined by a β-1,4′-glycosidic bond, has high commercial value in applications for the food/frozen foods, baking, brewing and beverage industries. Maltose is also a substrate for production of the non-caloric sugar sweetener, maltitol. High purity maltose or pure maltose is an active component of intravenous injection liquids for diabetic patients. Commercial processes for the production of syrup containing different levels of maltose content, i.e. <50% maltose (high conversion or low maltose syrup), 50-55% maltose (high maltose syrup), 70-75% maltose (very high maltose) and >80% maltose (ultra high maltose) have been established depending on the applications. A common factor for these processes is that they involve a dual enzyme process with two different steps, i.e. liquefaction and saccharification.

Historically, two enzyme steps are involved in the hydrolysis of starch to produce glucose or maltose syrups. The first step is a liquefaction step at high temperature, >95° C. and the second step is a saccharification step. In maltose production the second step is called malto-saccharification and usually takes place at a temperature at or below 60° C. In the liquefaction step, the insoluble starch granules are slurried in water, gelatinized with heat and hydrolyzed by a thermostable alpha-amylase (EC.3.2.1.1, α-1,4′-D-glucan glucanohydrolase) from Bacillus species, often in the presence of added calcium. Bacterial derived thermostable alpha-amylases from Bacillus licheniformis (for example, SPEZYME® FRED from DuPont-Genencor or Termamyl® L-120 from Novozymes), Bacillus stearothermophilus (for example SPEZYME® XTRA from DuPont-Genencor, Termamyl® SC, and Termamyl® SUPRA from Novozymes) or blends of Bacillus licheniformis and Bacillus stearothermophilus (for example Clearflow™ AA from DuPont-Genencor or Liquozyme® Supra from Novozymes) are used to first liquefy the starch at high temperature, >95° C. at pH 5.2-6.5 to a low DE (dextrose equivalent) soluble starch hydrolysate. In the malto-saccharification step, generally maltogenic enzymes such as a fungal alpha-amylase (for example, CLARASE® L from DuPont-Genencor or Fungamyl® 800L from Novozymes), a plant beta-amylase (for example, OPTIMALT® BBA from DuPont-Genencor or Betalase 1500L from Senson) are used at a much lower temperature to further hydrolyse the soluble starch hydrolysate. For maltose syrup containing greater than 60% maltose, a debranching enzyme like pullulanase (for example OPTIMAX® L-1000 from DuPont-Genencor, Promozyme® D2 from Novozymes or Promozyme® D6 from Novozymes) is added during malto-saccharification of liquefied starch.

The historical methods for production of maltose syrups using liquefaction and malto-saccharification require a high energy input for both heating the starch slurry to temperatures above 95° C. for liquefaction, and cooling to 60° C. for malto-saccharification. These methods are complex, involve multiple steps, multiple pH's and multiple operating temperatures. Another disadvantage of the high temperature is that it induces the formation of Maillard reaction products resulting in undesired color formation. Refined starch generally contains 0.2 to 0.5% proteins. A high temperature and high pH promotes the condensation of amino groups from proteins with reactive reducing group of sugars, generating reactive condensation products, the Maillard reaction products, which further polymerizes into high molecular weight polymers to produce undesired color. This undesired color requires further refining using active carbon treatment for its removal. In order to obtain an ultra high maltose syrup the alpha-amylase used in liquefaction needs to be inactivated in a so called ‘alpha kill’ step. This is generally done by passing the liquefied starch through a second jet-cooker at very high temperatures. Such a step is energy intensive and can cause additional Maillard product formation.

During liquefaction, the alpha-amylase hydrolyzes the starch. By each hydrolytic action of the enzyme, one α-1,4′-glycosidic bond in a starch molecule is broken resulting in 2 smaller glucose polymers, and consequently the formation of one additional reducing end and one additional non-reducing end.

During malto-saccharification, a beta-amylase releases consecutive maltose molecules from the non-reducing end of the glucose polymers formed during liquefaction. The action of the alpha-amylase during liquefaction has created numerous sites for the beta-amylase to attack the hydrolyzed starch. A beta-amylase cannot hydrolyse α-1,6′-glycosidic bonds and its action will stop at or near branch points. A debranching enzyme (e.g. pullulanase) does hydrolyse the α-1,6′-glycosidic bonds, and thus facilitates a more complete hydrolysis of liquefied starch by the beta-amylase, an effect that is well known in the industry. When a debranching enzyme hydrolyses an α-1,6′-glycosidic bond, it creates only one additional reducing end, not a non-reducing end.

Linear glucose polymers formed during liquefaction and by the action of pullulanase during malto-saccharification can contain an even or odd number of glucose molecules. Complete hydrolysis of linear glucose polymers with an even number of glucose molecules by a beta-amylase results in only maltose being formed. When a glucose polymer with an odd number of glucose molecules is hydrolyzed by a beta-amylase-maltotriose is left also, since it cannot be hydrolyzed by the beta-amylase.

It is known to be important to limit the formation of maltotriose when high and ultra high maltose hydrolysates are produced. For example, U.S. Pat. No. 4,917,916, U.S. Pat. No. 3,708,396, U.S. Pat. No. 3,804,715 describes the use of a low DE liquefact in order to obtain maltose syrups of sufficient purity. By using a low DE liquefact with a larger number of long glucose polymers, shorter glucose polymers and the consequent risk of generating odd-numbered polymers is reduced, resulting in a higher maltose content. DE's as low as 0.5 have been described for ultra high maltose syrup production. Unfortunately, working with a low DE liquefact is difficult due to a high viscosity and significant risk of retrogradation. The retrograded starch is resistant to hydrolysis by conventional malto-saccharification enzymes resulting in iodine positive maltose syrup (generally called “Blue Sac”). The iodine positive maltose syrup is not widely accepted in commerce because of its filtration problems associated with processing and affects on the quality of the final product.

To partially overcome these problems U.S. Pat. No. 4,917,916, U.S. Pat. No. 3,708,396, U.S. Pat. No. 3,804,715 also describe the use of a low Dry Solid (DS) content. Unfortunately, a low dry solid content has its own limitations, such as the large saccharification volumes needed and high energy input during evaporation. In theory, using a liquefact with a DE close to zero would result in the highest maltose content possible in malto-saccharification. This is because low DE limits the number of glucose polymers with an odd number of glucose residues as much as possible, resulting in highest maltose. In practice, however, such a low DE is not possible with historically-employed conventional processes involving a liquefaction step.

Other methods to produce ultra-high maltose syrups involve a normal 2 step hydrolysis reaction followed by some form of purification such as active carbon treatment (U.S. Pat. No. 4,294,623), chromatography (U.S. Pat. No. 4,487,198) and crystallization (U.S. Pat. No. 4,846,139). Such additional purification steps are complex and expensive.

Other methods to produce ultra-high maltose syrups involve a normal 2 step hydrolysis reaction and make use of a maltogenic alpha-amylase as a 4^{th} enzyme in addition to an alpha-amylase, beta-amylase and a debranching enzyme. Such methods are described in U.S. Pat. No. 6,346,400, U.S. Pat. No. 5,141,859, CA1052717.

Low temperature processes, where raw starch is hydrolyzed into maltose syrups have been described to partly overcome the described limitations of the liquefaction-saccharification processes. U.S. Pat. No. 5,188,956 disclosed a method for producing a malto-oligosaccharide syrup containing mainly maltose, maltotriose and glucose, using raw starch as substrate with an alpha-amylase from Bacillus stearothermophilus. The resulting syrup has around 50-55% maltose and a high content of maltotriose (30-36.5%). For the production of a high maltose syrup or ultra high maltose syrup this process will not be suitable. U.S. Pat. No. 6,361,809 also described a process for producing greater than 90% maltose using raw corn starch as substrate and an amylase with the enzyme classification EC.3.21.133 at a temperature below the starch gelatinization temperature. In this process, the reaction medium was continuously recycled via an Ultra Filtration (UF) module to remove smaller oligosaccharides, including maltose and glucose. The final yield of syrup from the UF module (permeate) had only 28% of initial starch weight and had a dry solid content of only 11%. Both the low DS, limited yield and the need for continuous recycling over an UF module are disadvantages of this process that will limit the applicability on industrial scale. GB1470325 describes the enzymatic hydrolysis of granular starch to glucose and maltose containing hydrolysates in a 1, 2 or 3 step process. In these processes no hydrolysates with a maltose content above 80% are described nor has the importance of a low alpha-amylase content heretofore been recognized.

Thus the prior art for producing high and ultra high maltose syrup has many disadvantages both from processing and economic point of view and warrants further process improvement.

### BRIEF SUMMARY OF THE CLAIMED INVENTION

The present teachings provide a method of making a high DP2 syrup containing at least 50% DP2 comprising; solubilizing a granular starch substrate at or below the initial gelatinization temperature with an exogenous alpha-amylase to form a mixture comprising dextrins; hydrolyzing the dextrins with a maltogenic enzyme to form the high DP2 syrup, wherein the ratio of alpha-amylase dose expressed as AAU/gds, to maltogenic enzyme dose expressed as DP degrees, is less than 8.

Additional methods, as well as compositions, are also provided.

### BRIEF DESCRIPTION OF THE FIGURES

FIG. 1 shows illustrative data compiled over several experiments according to the present teachings.

FIG. 2 shows illustrative data compiled over several experiments according to the present teachings.

FIG. 3 shows illustrative data compiled over several experiments according to the present teachings.

### DETAILED DESCRIPTION OF THE INVENTION

Here, we describe the unexpected finding that with a granular starch substrate an ultra low DE starch hydrolysate can be mimicked by treating the granular starch with a low activity dose of alpha-amylase in the presence of a high activity dose of a maltogenic enzyme. The treating can occur in a single step. A debranching enzyme can further facilitate a higher maltose content.

Without being limited to mechanism, the low concentration of alpha-amylase is believed to gradually solubilize the granular starch and the released glucose polymers are thereafter immediately hydrolyzed by the debranching enzyme and maltogenic enzyme. It was found that the ratio of beta-amylase over alpha-amylase is crucial for determining the maltose content in the resulting syrup and that under the appropriate conditions a maltose content of 90%, or greater, can be obtained.

This process further has the advantage that it can involve a one step process, can be performed at a single pH, can be performed at a single temperature, and can be accomplished with only one enzyme addition. This significantly simplifies the production process for ultra-high maltose syrups. Furthermore, no ‘alpha kill’ is required in this process (that is, the alpha-amylase need not be heat-inactivated through a time and energy-intensive process). In the processes of the present teachings, the DP3 content during the saccharification is kept low by using the appropriate ratio of alpha-amylase and beta-amylase.

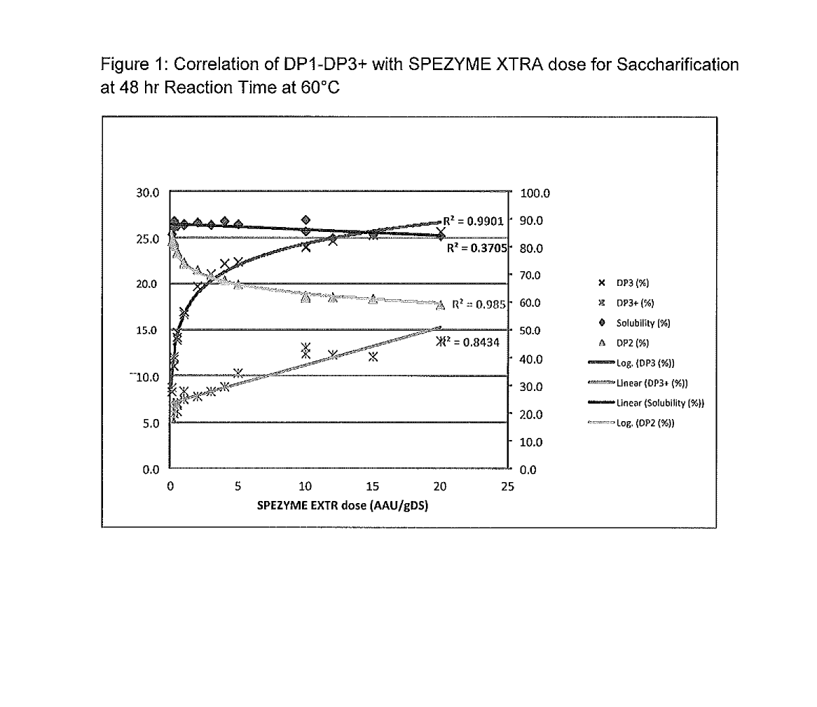

The present teachings describe the unexpected observation that a reduction of the alpha-amylase dose during maltose formation from granular starch or partially gelatinized starch resulted in an increased maltose content, a decreased DP3 and DP3+ content and resulted in higher solubilization. This is depicted in FIG. 1. In FIG. 1, the data from several experiments are combined to show the correlation between the alpha-amylase dose and the sugar composition of the reaction medium at 48 hours reaction time. FIG. 1 combines data from experiments done at 60° C. and 32% starting DS of Wheat Starch with SPEZYME® XTRA dosages varying between 0.1 and 20 AAU/gds. The beta-amylase dose (OPTIMALT® BBA) was constant at 0.63 DP °/gds, the pullulanase dose (OPTIMAX® L-1000) was varied between 0.5 and 3.0 ASPU/gds. FIG. 1 shows that there is a clear correlation between alpha-amylase dose and DP2, DP3 and DP3+. For solubilization there is a trend towards lower solubilization at higher alpha-amylase dose. The correlation between the sugar composition and alpha-amylase dose is not influenced by pullulanase dose in the range of 0.5-3.0.

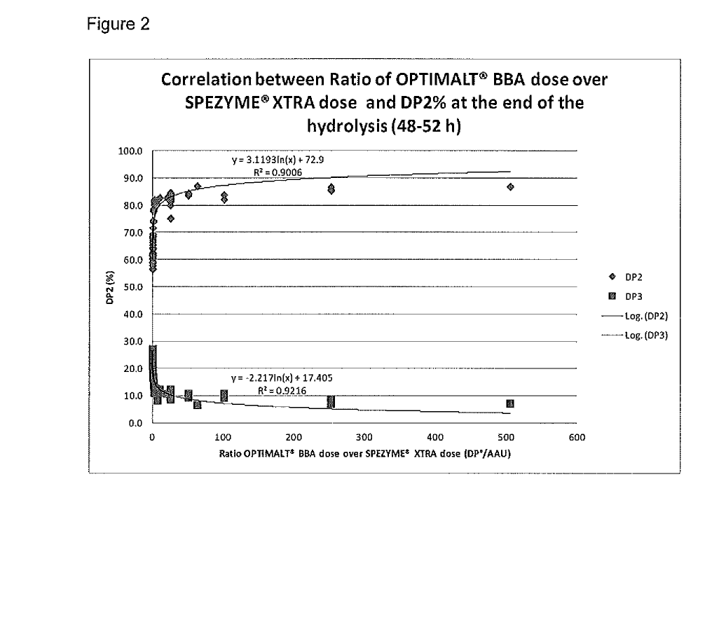

From these data we conclude that the low alpha-amylase likely slowly solubilizes the starch, which is immediately converted into maltose by the beta-amylase, assisted by the pullulanase. The situation created during the reaction is one where solubilized starch is present in low concentration and is exposed to a high enzyme dose of beta-amylase. Without being limited by theory, it is believed this is the reason why low DP3 is produced and high maltose content is possible. The data further shows that the ratio of beta-amylase over alpha-amylase units is important. The alpha-amylase dose can be increased, to facilitate solubilization, as long as the beta-amylase dose is increased the same. As long as the ratio of beta-amylase over alpha-amylase units remains the same, the maltose content in the final hydrolysate will be very similar. This can be seen from FIG. 2, where the ratio of beta-amylase activity units over alpha-amylase activity units is plotted against the maltose content in the hydrolysate after 48 hours. FIG. 2 combines data from experiments were only the pH was constant at 5.0, the dry solid varied between 10 and 40%, temperature for the combined experiments varied between 50 and 60° C., SPEZYME® XTRA dose varied between 0.01 and 20 AAU/gds, OPTIMALT® BBA dose varied between 0.19 and 10.08 DP°/gds, and OPTIMAX® L-1000 dose varied between 0.5 and 5.0 ASPU/gds.

Accordingly, in some embodiments the present teachings provide a method of making a high DP2 syrup containing at least 50% DP2 comprising; solubilizing a granular starch substrate at or below the initial gelatinization temperature with an exogenous alpha-amylase to form a mixture comprising dextrins; hydrolyzing the dextrins with a maltogenic enzyme present in stoichiometric excess relative to the exogenous alpha-amylase to form the high DP2 syrup.

In some embodiments, the present teachings provide a method of making a very high DP2 syrup containing at least 70% DP2 comprising; contacting a granular starch substrate at or below the initial gelatinization temperature with an exogenous alpha-amylase, and a maltogenic enzyme, wherein the ratio of alpha-amylase dose expressed as AAU/gds, to maltogenic enzyme dose expressed as DP degrees/gds, is 0.002-7.94. In some embodiments, this ratio is 0.005-7, or 0.02-4, or 0.01-2.

In some embodiments, the present teachings provide a method of making an ultra-high DP2 syrup containing at least 80% DP2 comprising; contacting a granular starch substrate at or below the initial gelatinization temperature with a pullulanase, an exogenous alpha-amylase, and a maltogenic enzyme, wherein the ratio of alpha-amylase dose expressed as AAU/gds, to maltogenic enzyme dose expressed as DP degrees/gds, is 0.002-0.42. In some embodiments, this ratio is 0.005-0.3, or 0.02-0.2, or 0.02-0.1.

In some embodiments of the present teachings, the reaction can be conducted at a temperature higher than the initial gelatinization temperature of a given starch. For example, in some embodiments the reaction is at 1, 2, 3, 3, 4, 5, 6, 7, 8, 9, 10, 11, 12, 13, 14, 15, 16, 17, 18, 19, or 20 degrees higher than the initial gelatinization temperature. In some embodiments, the reaction can be performed 1-5, 1-10, 5-10, 1-15, 5-15, or 1-20 degrees higher than the initial gelatinization temperature.

In some embodiments of the present teachings, the reaction can be conducted at a temperature lower than the initial gelatinization temperature of a given starch. For example, in some embodiments the reaction is at 1, 2, 3, 3, 4, 5, 6, 7, 8, 9, 10, 11, 12, 13, 14, 15, 16, 17, 18, 19, or 20 degrees lower than the initial gelatinization temperature. In some embodiments, the reaction can be performed 1-5, 1-10, 5-10, 1-15, 5-15, or 1-20 degrees lower than the initial gelatinization temperature.

In some embodiments, the present teachings provide a composition comprising an alpha-amylase and a maltogenic enzyme wherein the ratio of alpha-amylase dose expressed as AAU/gds, to maltogenic enzyme dose expressed as DP degrees/gds, is 0.002-7.94. In some embodiments, this ratio is 0.002-0.42, 0.005-7, or 0.02-4, or 0.01-2.

In some embodiments, following treatment with enzymes according to the present teachings, any residual undissolved starch can be subsequently used as a fermentation feedstock. For example the undissolved starch can be subjected to conventional liquefaction to form a liquefact that microbes can ferment to form various biochemicals, including for example ethanol, lactic acid, succinic acid, citric acid, monosodium glutamate, 1-3 propanediol, and the like. In some embodiments, the undissolved starch can be re-treated with the same enzymes used in a low temperature first treatment to create a syrup and/or fermentable substrate.

### General Techniques

Unless otherwise defined, all technical and scientific terms used have their ordinary meaning in the relevant scientific field. Singleton, et aL, Dictionary of Microbiology and Molecular Biology, 2d Ed., John Wiley and Sons, New York (1994), and Hale & Markham, Harper Collins Dictionary of Biology, Harper Perennial, NY (1991) provide the ordinary meaning of many of the terms describing the invention.

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., eds., 1994). Singleton et al., “Dictionary of Microbiology and Molecular Biology” 2nd ed., J. Wiley & Sons (New York, N.Y. 1994), and Baltz et al., “Manual of Industrial Microbiology and Biotechnology” 3^{rd} ed., (Washington, D.C.: ASM Press, 2010), provide one skilled in the art with a general guide to many of the terms used in the present application.
1) Carbohydrate composition by High Pressure Liquid Chromatographic (HPLC) method. The composition of the reaction products of oligosaccharides was measured by high pressure liquid chromatography (HPLC) using either method (a) or (b) as described below. Similar results are obtained for a given sample using either method (a) or method (b). Sample preparation of the slurry sample involves centrifugation for 5 minutes at 13000 rpm, dilution of the syrup to a 10% solution, and denaturation of enzymes by boiling for 10 minutes. Samples were cooled down and then filtered using a 0.22 μm disc filter (Titan Syringe Filter PTFE, 0.45 μm 30 mm) prior to HPLC analysis. In both methods, the HPLC column used separates saccharides by molecular weight. For example a designation of DP1 is a monosaccharide, such as glucose; a designation of DP2 is a disaccharide, such as maltose; a designation of DP3 is a trisaccharide, such as maltotriose and the designation “DP3⁺” is an oligosaccharide having a degree of polymerization (DP) of 4 or greater. Area percentages of the different saccharides (DP3+, DP3, DP2, DP1) are calculated by dividing the area of each individual saccharide by the total area of all saccharides.
   a. An HPLC system (Beckman System Gold 32 Karat Fullerton, Calif., USA) equipped with a HPLC column (Rezex RCM-Monosaccharides), maintained at 80° C. fitted with a refractive index (RI) detector was used. De-ionized water was used as the mobile phase at a flow rate of 0.6 ml per minute. Twenty microliter of 3.0% syrup solution was injected on to the column.
   b. An HPLC system (Prominence modular HPLC from Shimadzu Corporations; Kyoto, Japan) equipped with a HPLC column (Rezex RHM-monosaccharide H+(8%) phase from Phenomenex, Inc.; Torrance, Calif., USA) maintained at 85° C. was used. Ultrapure, demineralized water (MilliQ) was used as the mobile phase at a flow rate of 0.6 ml per minute. For each sample, 5 microliter of 10% syrup solution was injected on to the column.
2) One AAU of bacterial alpha-amylase activity is the amount of enzyme required to hydrolyze 10 mg of starch per min from 5% dry solids soluble Lintner starch solution containing 31.2 mM calcium chloride, at 60° C. and 6.0 pH buffered with 30 mM sodium acetate.
3) Beta-amylase activity determination in degrees Diastatic Power (DP°) Units. This assay is based on a 30-min hydrolysis of a starch substrate at pH 4.6 and 20° C. The reducing sugar groups produced on hydrolysis are measured in a titrimetric procedure using alkaline ferricyanide. One unit of diastase activity, expressed as degrees DP (DP°), is defined as the amount of enzyme, contained in 0.1 ml of a 5% solution of the sample enzyme preparation, that will produce sufficient reducing sugars to reduce 5 mL of Fehling's solution when the sample is incubated with 100 mL of the substrate for 1 hour at 20° C.
4) Percent solubilization of granular starch. Solubilization testing is done by sampling from the agitated slurry into 2.5 ml micro-centrifuge tube. The tube is spun for ˜4 minutes at 13,000 rpm and the refractive index of the supernatant is determined at 30° C. (RIₛᵤₚ). The total dry substance is determined by taking 1.5-2 ml of the starch slurry into a 2.5 ml spin tube, adding 1 drop of SPEZYME® FRED from a micro disposable-pipette then boiling 10 minutes. The tube is spun for ˜4 minutes at 13,000 rpm and the refractive index of the supernatant is determined at 30° C. (RIₜₒₜ). The dry substance of the supernatant (based on RIₛᵤₚ) and the whole sample (based on RIₜₒₜ) are determined using appropriate DE tables. Table for converting RIₛᵤₚ to DS is the 42 DE, Table E from the Critical Data Tables of the Corn Refiners Association, Inc. To convert RIₜₒₜ to DS, more than one table can be used and an interpolation between the 32 DE (Table D from the Critical Data Tables of the Corn Refiners Association, Inc.) and 42 DE tables employed. First an estimation of the solubilization is made by dividing the DS from the supernatant by the starting DS*1.05. This estimated solubilization is used for the interpolation between the DS obtained via the 42DE and 32DE table. Solubilization is defined as the dry substance of the supernatant divided by the total dry substance times 100. This value is then corrected to compensate for the impact of remaining granular starch (eg—the undissolved starch which takes up space and thus distorts the calculations, comprising the residual starch in the reaction medium).
5) One Acid Stable Pullulanase Unit (ASPU) is the amount of enzyme which liberates one equivalent reducing potential as glucose per minute from pullulan at pH 4.5 and a temperature of 60° C.
6) Maltogenic Amylase Units (MAA). This method is a UV absorption method that utilizes maltotriose as soluble substrate. The Maltogenic amylase converts the maltotriose into maltose and glucose. Being catalyzed by Gluc-DH (Glucose Dehydrogenase) the glucose will reduce the NAD (Nicotin Adenin Dinucleotide) to NADH. Formation of NADH (a measure for an equivalent to glucose) can be determined with UV detection. As will be appreciated by one of ordinary skill in the art, a maltogenic amylase dose expressed in MAA units can readily be tested in an AAU assay to determine the related AAU units, as well as tested in an assay to determine the related DP° units.
7) Fungal alpha-amylase (SKB) units. This method is used to determine fungal amylase activity. The assay uses p-nitrophenyl maltoheptoside substrate with the non-reducing terminal sugar chemically blocked. Alpha-glucosidase and a glucoamylase are used as coupling enzymes. The blocked terminal sugar prevents attack by glucoamylase. The rate of p-nitrophenyl release is proportional to fungal amylase activity and is monitored at 410 nm. As will be appreciated by one of ordinary skill in the art, a fungal alpha-amylase dose expressed in SKB units can readily be tested in an AAU assay to determine the related AAU units, as well as tested in an assay to determine the related DP° units.

### DEFINITIONS

The term “granular starch” refers to uncooked (raw) starch, which has not been subject to gelatinization.

The term “starch gelatinization” means solubilization of starch molecules to form a viscous suspension.

The term “initial gelatinization temperature” refers to the lowest temperature at which gelatinization of a starch substrate begins. The exact temperature can be readily determined by the skilled artisan, and depends upon the specific starch substrate and further may depend on the particular variety of plant species from which the starch is obtained and the growth conditions of the plant. According to the present teachings, the initial gelatinization temperature of a given starch is the temperature at which birefringence is lost in 5% of the starch granules using the method described by Gorinstein. S. and Lii. Cl., Starch/Stark, Vol 44 (12) pp. 461-466 (1992). The initial starch gelatinization temperature ranges for a number of granular starches, which may be used in accordance with the processes herein include barley (52-59° C.), wheat (58-64° C.), rye (57-70° C.), corn (62-72° C.), high amylose corn (67-80° C.), rice (68-77° C.), sorghum (68-77° C.), potato (58-68° C.), tapioca (59-69° C.) and sweet potato (58-72° C.) (Swinkels, pg. 32-38 in STARCH CONVERSION TECHNOLOGY, Eds Van Beynum et al., (1985) Marcel Dekker Inc. New York and The Alcohol Textbook 3.sup.rd ED. A Reference for the Beverage, Fuel and Industrial Alcohol Industries, Eds Jacques et al., (1999) Nottingham University Press, UK). Gelatinization involves melting of crystalline areas, hydration of molecules and irreversible swelling of granules. The gelatinization temperature occurs in a range for a given grain because crystalline regions vary in size and/or degree of molecular order or crystalline perfection. STARCH HYDROLYSIS PRODUCTS Worldwide Technology, Production, and Applications (eds/Shenck and Hebeda, VCH Publishers, Inc, New York, 1992) at p. 26.

The term “DE” or “dextrose equivalent” is an industry standard for measuring the concentration of total reducing sugars, calculated as D-glucose on a dry weight basis. Unhydrolyzed granular starch has a DE that is essentially 0 and D-glucose has a DE of 100.

The term “starch substrate” refers to granular starch or liquefied starch where starch can be refined starch, whole ground grains or fractionated grains. The starch substrate can arise from any of a variety of sources, including corn, wheat, barley, rye, triticale, rice, oat, beans, banana, potato, sweet potato, sorghum, legumes, cassava, millet, potato, or tapioca.

The term “slurry” is an aqueous mixture containing unsolubilized starch granules.

The term “liquefied starch” refers to starch which has gone through solubilization process using conventional starch liquefaction process.

The term “maltose syrup” refers to an aqueous composition containing maltose solids. Various levels of maltose syrup can be defined, for example <50% maltose refers to a low maltose syrup, 50-55% maltose refers to a high maltose syrup, 70-75% maltose refers to a very high maltose, and >80% maltose refers to an ultra high maltose syrup.

The term “dry solids content (DS)” refers to the total solids (dissolved and undissolved) of a slurry (in %) on a dry weight basis. At the onset, “initial DS” refers to the dry solids in the slurry at time zero. As the hydrolysis reaction proceeds, the portion of DS that are dissolved can be referred to as “Syrup DS” as well as “Supernatant DS”.

An “alpha-amylase” (E.C. class 3.2.1.1) is an enzyme that catalyzes the hydrolysis of α-1,4′-D-glucosidic linkages. These enzymes have also been described as those effecting the exo or endo hydrolysis of α-1,4′-D-glucosidic linkages in polysaccharides containing α-1,4′-linked D-glucose units. Another term used to describe these enzymes is glycogenase. Exemplary enzymes include α-1,4′-glucan-4-glucanohydrase glucanohydrolase. In some of the embodiments encompassed by the invention, the alpha-amylase is an enzyme having an E.C. number, E.C. 3.2.1.1. In some embodiments, the alpha-amylase is a thermostable bacterial alpha-amylase. Suitable alpha-amylases may be naturally occurring as well as recombinant and mutant alpha-amylases. In particularly preferred embodiments, the alpha-amylase is derived from a Bacillus species. Preferred Bacillus species include B. subtilis, B. stearothermophilus, B. lentus, B. licheniformis, B. coagulans, and B. amyloliquefaciens (U.S. Pat. No. 5,763,385; U.S. Pat. No. 5,824,532; U.S. Pat. No. 5,958,739; U.S. Pat. No. 6,008,026 and U.S. Pat. No. 6,361,809). Particularly, preferred alpha-amylases are derived from Bacillus strains B. stearothermophilus, B. amyloliquefaciens and B. licheniformis. Also reference is made to strains having ATCC 39709; ATCC 11945; ATCC 6598; ATCC 6634; ATCC 8480; ATCC 9945A and NCIB 8059. Commercially available alpha-amylases contemplated for use in the methods of the invention include; SPEZYME® AA; SPEZYME® XTRA; SPEZYME® FRED; GZYME® G997 from DuPont-Genencor and Termamyl® 120-L, Termamyl® LC, Termamyl® SC, Liquozyme® SC and Liquozyme® SUPRA from Novozymes.

The term “beta-amylase” (E.C. class 3.2.1.2) refers to enzymes that catalyze the hydrolysis of α-1,4-D-glucosidic linkages in polysaccharides so as to remove successive maltose units from the non-reducing ends of the chains. Other terms used to describe these enzymes are α-1,4-glucan maltohydrolases.

The term “maltogenic enzyme” refer to enzymes that are capable of producing significant amounts of maltose from starch or hydrolyzed starch. There are several enzymes falling under this definition. Some examples are:
1) Fungal alpha-amylases include those obtained from filamentous fungal strains including but not limited to strains of Aspergillus (e.g., A. Niger, A. kawachi, and A. oryzae); Trichoderma sp. (e.g. Trichoderma reesei alpha-amylase, disclosed in EP 2132307), Rhisopus sp., Mucor sp., and Penicillium sp. Commercial fungal alpha-amylase from Aspergillus oryzae are CLARASE® L from DuPont-Genencor and Fungamyl® 800L from Novozymes.
2) Acid stable fungal amylase from Aspergillus niger (For example, from Shin Nihon Chemicals).
3) Beta-amylases are found in plant materials like wheat, barley, rye, sorghum, soy, sweet potato, rice and microorganisms like Bacillus cereus, Bacillus polymixa, Bacillus megaterium, Arabidopsis thaliana. The most common commercial beta-amylases are derived from barley and are sold under trade the names OPTIMALT® BBA by DuPont-Genencor and Betalase 1500L by Senson. A commercial soy beta-amylase is β-amylase#1500S from Nagase ChemteX Corporation.
4) Maltogenic amylases (E.C. 3.2.1.133) are produced by microorganisms Bacillus subtilis, Geobacillus stearothermophilus, Bacillus thermoalkalophilus, Lactobacillus gasseri, Thermus sp. Commercial maltogenic amylases include but not limited to Maltogenase® L from Novozymes, Veron® XTENDER from AB Enzymes and MAX-LIFE™ P100 from DuPont-Danisco.

The term “debranching enzymes” refer to enzymes that are capable of catalyzing the hydrolysis of α-1,6-D-glucosidic linkages in pullulan and in amylopectin and glycogen, and the alpha- and beta-limit dextrins of amylopectin and glycogen. Examples of debranching enzymes include pullulanases (E.C. 3.2.1.41) and iso-amylases (EC 3.2.1.68). Pullulanases are generally secreted by a Bacillus species. For example, Bacillus deramificans (U.S. Pat. No. 5,817,498; 1998), Bacillus acidopullulyticus (European Patent #0 063 909) and Bacillus naganoensis (U.S. Pat. No. 5,055,403). Enzymes having pullulanase activity used commercially are produced for examples, from Bacillus species (trade name OPTIMAX® L-1000 from DuPont-Genencor and Promozyme® D2 from Novozymes). Other examples of debranching enzymes include (but not limited to) iso-amylase from Sulfolobus solfataricus, Pseudomonas sp. and thermostable pullulanase from Fervidobacterium nodosum (eg WO2011076123). The isoamylase from Pseudomonas sp. is available as purified enzyme from Megazyme International.

The term “hydrolysis of starch” refers to the cleavage of glucosidic bonds with the addition of water molecules.

The term “degree of polymerization (DP)” refers to the number (n) of anhydroglucopyranose units in a given saccharide. Examples of DP1 are the monosaccharides, such as glucose and fructose. Examples of DP2 are the disaccharides, such as maltose and sucrose. A DP3⁺ (>DP3) denotes polymers with a degree of polymerization equal or greater than 4.

The term “contacting” refers to the placing of the respective enzymes in sufficiently close proximity to the respective substrate to enable the enzymes to convert the substrate to the end product. Those skilled in the art will recognize that mixing solutions of the enzyme with the respective substrates can effect contacting.

The term “solubilization” is used in a broad meaning as to bring to a solution by any means.

The term “comprising” and its cognates are used in their inclusive sense; that is, equivalent to the term “including” and its corresponding cognates.

Unless defined otherwise herein, all technical and scientific terms used herein have the same meaning as commonly understood by one of ordinary skill in the art to which this invention pertains.

The term “temperature staging” is used to refer to use of more than one temperature to carry out a reaction, preferably conversion of starch to maltose. The temperatures may range from about 4° C. to about 99° C. In some embodiments, the temperatures may range from 25° C. to 95° C. In some embodiments, the temperatures may range from 37° C. to 70° C. In some embodiments, the temperatures may range from 50° C. to 60° C.

As used herein, the singular terms “a,” “an,” and “the” include the plural reference unless the context clearly indicates otherwise.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The invention can be further understood by reference to the following examples, which are provided by way of illustration and are not meant to be limiting.

### EXAMPLES

### Example 1

In this experiment, the influence of SPEZYME® XTRA dose on the solubilization of granular (undissolved) wheat starch (Roquette wheat bag starch) in the presence of OPTIMALT® BBA and OPTIMAX® L-1000, as well as the influence on resulting sugar composition of the hydrolysate was studied.

A 32% DS aqueous slurry of wheat starch was incubated at pH 5.0 and 60° C. with 0.63 DP°/gds OPTIMALT® BBA, 0.5 ASPU/gds OPTIMAX® L-1000 and SPEZYME® XTRA with a dose varying between 0.1 and 20 AAU/gds for 48-50 hours. The slurry was constantly stirred and samples were taken at different time intervals for determination of starch solubilization and sugar composition. The enzyme dosages and results are shown in Table 1.

Effect of SPEZYME® XTRA, at constant OPTIMALT® BBA and constant OPTIMAX® L-1000 dose on the solubilization of granular wheat starch at pH 5.0 and 60° C. and the resulting sugar composition.

| SPEZYME ® | OPTIMALT ® | OPTIMAX ® | | | | | | |
|---|---|---|---|---|---|---|---|---|
| XTRA | BBA | L-1000 | Time | Solubilization | DP1 | DP2 | DP3 | DP3+ |
| AAU/g DS | DP°/gdS | ASPU/gdS | hours | % | % | % | % | % |
| 20 | 0.63 | 0.5 | 3.5 | 74.0 | 0.9 | 50.5 | 22.4 | 26.2 |
| | | | 20 | 82.7 | 1.2 | 59.7 | 25.5 | 13.6 |
| | | | 28 | 83.8 | 1.3 | 59.7 | 25.7 | 13.3 |
| | | | 44 | 84.3 | 1.4 | 59.0 | 25.8 | 13.8 |
| | | | 48 | 84.0 | 1.4 | 59.1 | 25.7 | 13.8 |
| 15 | 0.63 | 0.5 | 3.5 | 71.9 | 0.8 | 51.4 | 21.7 | 26.2 |
| | | | 20 | 81.0 | 1.0 | 61.2 | 25.0 | 12.8 |
| | | | 28 | 83.1 | 1.2 | 61.5 | 25.2 | 12.1 |
| | | | 44 | 84.0 | 1.2 | 61.1 | 25.2 | 12.4 |
| | | | 48 | 84.5 | 1.3 | 61.3 | 25.3 | 12.1 |
| 12 | 0.63 | 0.5 | 3.5 | 72.7 | 0.7 | 51.5 | 20.9 | 26.9 |
| | | | 20 | 81.5 | 1.1 | 61.7 | 24.3 | 12.8 |
| | | | 28 | 82.7 | 1.1 | 62.2 | 24.6 | 12.0 |
| | | | 44 | 83.0 | 1.1 | 61.9 | 24.7 | 12.3 |
| | | | 48 | 83.2 | 1.1 | 61.9 | 24.7 | 12.3 |
| 10 | 0.63 | 0.5 | 3.5 | 77.3 | 0.8 | 52.2 | 20.4 | 26.6 |
| | | | 20 | 86.2 | 1.1 | 62.1 | 23.8 | 13.0 |
| | | | 24 | 87.1 | 1.2 | 62.2 | 23.9 | 12.8 |
| | | | 28 | 87.8 | 1.1 | 62.2 | 23.9 | 12.8 |
| | | | 44 | 89.3 | 1.3 | 61.7 | 24.0 | 13.1 |
| | | | 48 | 89.8 | 1.3 | 61.6 | 23.9 | 13.1 |
| 5 | 0.63 | 0.5 | 3.5 | 74.4 | 0.6 | 55.0 | 18.0 | 26.4 |
| | | | 20 | 83.9 | 0.8 | 65.8 | 21.8 | 11.7 |
| | | | 24 | 85.1 | 0.7 | 66.3 | 22.0 | 11.0 |
| | | | 28 | 86.2 | 0.8 | 66.6 | 22.1 | 10.5 |
| | | | 44 | 87.9 | 1.0 | 66.5 | 22.3 | 10.3 |
| | | | 48 | 88.2 | 0.8 | 66.5 | 22.4 | 10.3 |
| 4 | 0.63 | 0.5 | 3.5 | 76.9 | 0.7 | 54.8 | 17.4 | 27.0 |
| | | | 20 | 85.6 | 0.8 | 66.3 | 21.3 | 11.6 |
| | | | 28 | 86.6 | 1.1 | 67.0 | 21.5 | 10.4 |
| | | | 44 | 88.6 | 0.9 | 68.0 | 22.1 | 9.1 |
| | | | 48 | 89.3 | 1.0 | 68.0 | 22.2 | 8.9 |
| 3 | 0.63 | 0.5 | 3.5 | 75.7 | 0.9 | 55.9 | 16.0 | 27.1 |
| | | | 20 | 84.6 | 0.7 | 67.9 | 20.2 | 11.2 |
| | | | 28 | 85.9 | 0.8 | 69.0 | 20.4 | 9.8 |
| | | | 44 | 87.5 | 0.9 | 69.6 | 21.0 | 8.5 |
| | | | 48 | 88.0 | 1.2 | 69.4 | 21.1 | 8.3 |
| 2 | 0.63 | 0.5 | 3.5 | 74.8 | 0.8 | 57.3 | 14.3 | 27.6 |
| | | | 20 | 84.5 | 1.0 | 69.8 | 18.7 | 10.6 |
| | | | 28 | 85.9 | 1.2 | 70.5 | 19.1 | 9.3 |
| | | | 44 | 88.1 | 0.6 | 71.9 | 19.6 | 7.9 |
| | | | 48 | 88.8 | 0.7 | 71.7 | 19.7 | 7.8 |
| 1 | 0.63 | 0.5 | 3.5 | 72.6 | 0.8 | 59.6 | 10.9 | 28.6 |
| | | | 20 | 82.8 | 1.0 | 72.7 | 16.0 | 10.3 |
| | | | 28 | 85.1 | 1.1 | 73.7 | 16.4 | 8.8 |
| | | | 44 | 87.6 | 1.2 | 74.4 | 16.9 | 7.5 |
| | | | 48 | 88.3 | 1.1 | 74.4 | 17.0 | 7.5 |
| 0.5 | 0.63 | 0.5 | 5 | 75.6 | 0.7 | 64.6 | 8.8 | 25.8 |
| | | | 21.25 | 83.0 | 0.9 | 76.3 | 13.1 | 9.7 |
| | | | 24 | 83.6 | 0.9 | 76.9 | 13.3 | 8.9 |
| | | | 28 | 84.3 | 0.9 | 77.4 | 13.6 | 8.1 |
| | | | 46 | 87.4 | 0.9 | 78.1 | 14.2 | 6.9 |
| | | | 50 | 87.6 | 1.0 | 77.8 | 14.2 | 7.0 |
| 0.25 | 0.63 | 0.5 | 5 | 77.3 | 0.6 | 63.3 | 6.4 | 29.6 |
| | | | 21.25 | 83.0 | 0.9 | 77.1 | 10.3 | 11.7 |
| | | | 24 | 83.4 | 0.9 | 77.9 | 10.6 | 10.5 |
| | | | 28 | 83.7 | 0.8 | 79.2 | 11.0 | 9.0 |
| | | | 46 | 86.3 | 0.9 | 80.3 | 11.7 | 7.1 |
| | | | 50 | 87.0 | 0.9 | 80.4 | 11.8 | 6.8 |
| 0.1 | 0.63 | 0.5 | 5 | 69.7 | 0.4 | 66.2 | 3.9 | 29.5 |
| | | | 21.25 | 81.0 | 0.9 | 78.4 | 6.9 | 13.8 |
| | | | 24 | 81.7 | 0.8 | 78.6 | 7.1 | 13.5 |
| | | | 28 | 82.0 | 0.7 | 80.2 | 7.4 | 11.6 |
| | | | 46 | 84.4 | 0.9 | 81.8 | 8.2 | 9.2 |
| | | | 50 | 84.5 | 0.9 | 82.0 | 8.3 | 8.8 |

Table 1 shows that at decreasing SPEZYME® XTRA dose, the maltose content increases from 59.7% at the highest dose to 82% at the lowest dose. The DP3 and DP3+ content decreases with decreasing alpha-amylase dose and DP1 content remains constant around 1.0%. There is a trend towards higher solubilization at lower alpha-amylase dose.

In this example, the difference between the individual experiments is not only the alpha-amylase dose. Rather, due to the constant beta-amylase dose also the ratio of beta-amylase activity over alpha-amylase activity is changed. With an increasing ratio of beta-amylase over alpha-amylase activity, the DP2 content increases and DP3 and DP3+ content decreases.

This example shows that lowering the alpha-amylase dose is beneficial for production of an ultra high maltose product under the present invention.

### Example 2

In this experiment the influence of OPTIMALT® BBA dose on the solubilization of granular wheat starch (Roquette wheat bag starch) in the presence of SPEZYME® XTRA and OPTIMAX® L-1000, as well as the influence on resulting sugar composition of the hydrolysate was studied at 60° C.

A 32% DS aqueous slurry of wheat starch was incubated at pH 5.0 and 60° C. with 0.63, 1.2 and 2.4 DP°/gds OPTIMALT® BBA, 1.0 ASPU/gds OPTIMAX® L-1000 and 0.50 AAU/gds SPEZYME® XTRA for 50 hours. The slurry was constantly stirred and samples were taken at different time intervals for determination of % starch solubilization and sugar composition. The enzyme dose and results are shown in Table 2.

TABLE 2
Effect of OPTIMALT ® BBA dose at constant OPTIMAX ® L-1000 and SPEZYME ® XTRA
dose on the solubilization of granular wheat starch at pH 5.0 and 60° C. and the resulting
sugar composition.

| SPEZYME ® | OPTIMALT ® | OPTIMAX ® | | | | | | |
|---|---|---|---|---|---|---|---|---|
| XTRA | BBA | L-1000 | Time | Solubilization | DP1 | DP2 | DP3 | DP3+ |
| AAU/g DS | DP°/gds | ASPU/gds | hours | % | % | % | % | % |
| 0.5 | 0.63 | 1 | 5 | 75.3 | 0.8 | 69.8 | 10.9 | 18.5 |
| | | | 21.25 | 82.6 | 0.9 | 78.5 | 14.1 | 6.4 |
| | | | 24 | 83.8 | 0.9 | 78.9 | 14.3 | 5.9 |
| | | | 28 | 84.4 | 0.9 | 79.0 | 14.4 | 5.8 |
| | | | 46 | 87.3 | 0.9 | 78.4 | 14.7 | 6.0 |
| | | | 50 | 87.6 | 0.9 | 78.1 | 14.8 | 6.2 |
| 0.5 | 1.2 | 1 | 5 | 76.8 | 0.4 | 74.0 | 9.6 | 16.0 |
| | | | 21.25 | 84.2 | 0.9 | 80.8 | 12.4 | 5.8 |
| | | | 24 | 85.0 | 0.6 | 81.2 | 12.6 | 5.7 |
| | | | 28 | 85.9 | 0.9 | 80.9 | 12.7 | 5.5 |
| | | | 46 | 88.8 | 1.0 | 80.4 | 12.9 | 5.7 |
| | | | 50 | 89.1 | 1.0 | 80.2 | 13.0 | 5.8 |
| 0.5 | 2.4 | 1 | 5 | 77.8 | 0.9 | 76.2 | 8.8 | 14.2 |
| | | | 21.25 | 84.2 | 1.0 | 82.2 | 11.3 | 5.5 |
| | | | 24 | 85.1 | 0.9 | 82.4 | 11.3 | 5.4 |
| | | | 28 | 85.5 | 0.9 | 82.4 | 11.4 | 5.3 |
| | | | 46 | 87.6 | 1.0 | 81.8 | 11.6 | 5.6 |
| | | | 50 | 87.9 | 0.9 | 81.6 | 11.7 | 5.8 |

Table 2 shows that increasing the OPTIMALT® BBA dose only marginally influences the % solubilization of the granular starch in 50 hours. Solubilization ranges from 87.6-89.1%. The DP2 content is increased from 78.9% at the lowest dose to 82.4% at the highest dose, and DP3 content decreases with higher OPTIMALT® BBA dose. DP3+ is decreased especially early in the reaction and DP1 is practically unaffected. This faster decrease in DP3+ content shows that the beta-amylase quickly degrades the DP3+ that was released from the granular starch by the action of the alpha-amylase.

In this example, the difference between the individual experiments is not only the beta-amylase dose. Due to the constant alpha-amylase dose also the ratio of beta-amylase activity over alpha-amylase activity is changed. With an increasing ratio of beta-amylase over alpha-amylase activity, the DP2 content increases and DP3 and DP3+ content decreases.

This example shows that increasing the beta-amylase dose is beneficial for production of an ultra high maltose product under the present invention.

### Example 3

In this experiment the influence of OPTIMAX® L-1000 dose on the solubilization of granular wheat starch (Roquette wheat bag starch) in the presence of SPEZYME® XTRA and OPTIMALT® BBA, as well as the influence on resulting sugar composition of the hydrolysate was studied at 60° C.

A 32% DS aqueous slurry of wheat starch was incubated at pH 5.0 and 60° C. for 50 hours with 0.63 DP °/gds OPTIMALT® BBA, 0.50 AAU/gds SPEZYME® XTRA and OPTIMAX® L-1000 with a dose varying between 0.5 and 3.0 ASPU/gds. The slurry was constantly stirred and samples were taken at different time intervals for determination of starch solubilization and sugar composition. The enzyme dose and results are shown in Table 3.

TABLE 3
Effect of OPTIMAX ® L-1000 dose at constant OPTIMALT ® BBA and SPEZYME ® XTRA
dose on the solubilization of granular wheat starch at pH 5.0 and 60° C. and the resulting
sugar composition.

| SPEZYME ® | OPTIMALT ® | OPTIMAX ® | | | | | | |
|---|---|---|---|---|---|---|---|---|
| XTRA | BBA | L-1000 | Time | Solubilization | DP1 | DP2 | DP3 | DP3+ |
| AAU/g DS | DP°/gds | ASPU/gds | Hours | % | % | % | % | % |
| 0.25 | 0.63 | 0.5 | 5 | 77.3 | 0.6 | 63.3 | 6.4 | 29.6 |
| | | | 21.25 | 83.0 | 0.9 | 77.1 | 10.3 | 11.7 |
| | | | 24 | 83.4 | 0.9 | 77.9 | 10.6 | 10.5 |
| | | | 28 | 83.7 | 0.8 | 79.2 | 11.0 | 9.0 |
| | | | 46 | 86.3 | 0.9 | 80.3 | 11.7 | 7.1 |
| | | | 50 | 87.0 | 0.9 | 80.4 | 11.8 | 6.8 |
| 0.25 | 0.63 | 1 | 5 | 74.9 | 0.7 | 70.1 | 7.9 | 21.3 |
| | | | 21.25 | 83.1 | 0.9 | 80.8 | 11.3 | 7.0 |
| | | | 24 | 83.7 | 0.9 | 81.0 | 11.5 | 6.6 |
| | | | 28 | 85.1 | 0.5 | 81.8 | 11.7 | 6.1 |
| | | | 46 | 88.1 | 0.9 | 81.7 | 12.1 | 5.3 |
| | | | 50 | 88.9 | 0.9 | 81.6 | 12.1 | 5.3 |
| 0.25 | 0.63 | 2 | 5 | 72.8 | 0.8 | 73.6 | 8.2 | 17.4 |
| | | | 21.25 | 82.6 | 0.5 | 82.9 | 10.7 | 5.9 |
| | | | 24 | 83.7 | 0.9 | 82.6 | 10.8 | 5.7 |
| | | | 28 | 84.6 | 0.9 | 82.7 | 10.9 | 5.5 |
| | | | 46 | 89.0 | 0.4 | 82.6 | 11.2 | 5.8 |
| | | | 50 | 89.5 | 0.9 | 82.1 | 11.1 | 5.9 |
| 0.25 | 0.63 | 3 | 5 | 72.1 | 0.9 | 75.4 | 8.6 | 15.1 |
| | | | 21.25 | 81.8 | 1.0 | 82.5 | 10.7 | 5.7 |
| | | | 24 | 82.4 | 1.0 | 82.7 | 11.0 | 5.4 |
| | | | 28 | 83.7 | 0.9 | 82.5 | 11.0 | 5.5 |
| | | | 46 | 87.6 | 0.9 | 81.7 | 11.1 | 6.3 |
| | | | 50 | 88.5 | 1.0 | 81.4 | 11.1 | 6.5 |

Table 3 shows that the solubilization of granular wheat starch in 50 hours is not significantly affected by the OPTIMAX® L-1000 dose. DP2 formation is faster with increasing OPTIMAX® L-1000 dose and peak DP2 is increasing with increasing dose up to 2 ASPU/gds. A dose of 2 ASPU/gds of OPTIMAX® L-1000 seems optimal as higher dose does not improve DP2 or DP3. Final DP2 content at 50 hour reaction time are nearly the same for all OPTIMAX® L-1000 dosages. DP3 is not affected significantly and DP3+ is lower early in the reaction with higher OPTIMAX® L-1000 dose, but similar at 50 hours reaction.

This example shows that increasing OPTIMAX® L-1000 is beneficial for speeding up the reaction and increasing peak DP2.

### Example 4

The experiment was carried out to determine the effect of dry solids content, i.e.10%, 20%, 30% and 40% DS during incubation of granular wheat starch (Roquette wheat bag starch) with SPEZYME® XTRA, OPTIMAX® L-1000 and OPTIMALT® BBA. In a typical experiment, aqueous wheat starch slurry was made at different dry solids content, i.e. 10%, 20%, 30%, and 40% DS. The pH was adjusted to pH 5.0. Enzymes, SPEZYME® XTRA (0.1 AAU/gds), OPTIMAX® L-1000 (2.0 ASPU/gds) and OPTIMALT® BBA (2.52 DP °/gds) were added and incubated in a water bath maintained at 55° C.

The slurry was constantly stirred and samples were taken at different time intervals for determination of % starch solubilization and sugar composition. The enzyme dose and results are shown in Table 4.

TABLE 4
Effect of dry solids content during incubation of granular wheat starch
with SPEZYME ® XTRA (0.1 AAU/gds), OPTIMAX ® L1-000
(2.0 ASPU/gds) and OPTIMALT ® BBA (2.52 DP°/gds), pH 5.0, 55° C.
on the percent starch solubilization and sugar composition.

| DS | Time | Solubilization | DP1 | DP2 | DP3 | DP3+ |
|---|---|---|---|---|---|---|
| % | hours | % | % | % | % | % |
| 10 | 3 | 41.6 | 1.0 | 83.4 | 7.5 | 8.1 |
| | 20 | 76.2 | 1.0 | 85.5 | 10.4 | 3.1 |
| | 28 | 80.5 | 1.1 | 85.2 | 10.9 | 2.9 |
| | 44 | 85.1 | 1.1 | 83.7 | 11.2 | 4.0 |
| | 48 | 85.8 | 1.0 | 84.0 | 11.2 | 3.7 |
| 20 | 3 | 39.4 | 1.0 | 84.6 | 7.7 | 6.8 |
| | 20 | 68.6 | 1.1 | 84.3 | 11.1 | 3.5 |
| | 28 | 72.3 | 1.1 | 84.0 | 11.4 | 3.6 |
| | 44 | 77.4 | 1.1 | 83.4 | 11.7 | 3.8 |
| | 48 | 78.4 | 1.1 | 83.2 | 11.8 | 3.9 |
| 30 | 3 | 37.1 | 1.0 | 84.0 | 7.8 | 7.2 |
| | 20 | 59.3 | 1.1 | 84.4 | 9.9 | 4.6 |
| | 28 | 62.7 | 1.1 | 83.8 | 10.2 | 5.0 |
| | 44 | 67.5 | 1.1 | 82.7 | 10.6 | 5.6 |
| | 48 | 68.5 | 1.1 | 82.6 | 10.6 | 5.7 |
| 40 | 3 | 29.7 | 1.1 | 84.3 | 8.3 | 6.3 |
| | 20 | 52.5 | 1.1 | 83.7 | 10.2 | 4.9 |
| | 28 | 55.2 | 1.1 | 83.3 | 10.4 | 5.2 |
| | 44 | 58.7 | 1.1 | 82.6 | 10.6 | 5.8 |
| | 48 | 59.6 | 1.1 | 82.3 | 10.6 | 6.0 |

Solubilization of granular wheat starch by the enzyme composition of the present teachings decreased with increasing dry solids content. The solubilization of granular starch reached 85.8% at 10% dry solids but it was only 59.6% at 40% dry solids. The sugar composition is only marginally affected by the variation in % DS. DP2 at the start of the reaction varies between 83.4% and 84.6% and after 48 hours hydrolysis between 82.3% and 84%. DP3 at the start of the hydrolysis are very similar (7.5-8.3%) and range between 10.6% and 11.8% after 48 hours hydrolysis. There is no clear correlation between % DP3 and % DS. DP3 content is highest for 20% DS and lowest for 30% and 40% DS. Similar variations are seen for DP3+, where the highest value is seen for 30% and 40% DS and the lowest for 10% and 20% DS. There is no variation in DP1 with varying dry solid content.

This example shows that the DS can be varied without influencing the percentage of maltose in the resulting hydrolysate. Naturally the DS does influence the absolute maltose content (g/l) of the syrup.

### Example 5

In this experiment the importance of OPTIMAX® L-1000 addition during the hydrolysis of granular wheat starch (Roquette wheat bag starch) in the presence of SPEZYME® XTRA and OPTIMALT® BBA for starch solubilization and resulting sugar composition was tested at 55° C. and pH 5.0.

A 32% DS aqueous slurry of wheat starch was incubated at pH 5.0 and 55° C. with 2.52 DP°/gds OPTIMALT® BBA, 0.1 AAU/gds SPEZYME® XTRA with and without 2.0 ASPU/gds OPTIMAX® L-1000 for 48 hours. The slurry was constantly stirred and samples were taken at different time intervals for determination of % starch solubilization and sugar composition. The solubilization and sugar composition results are shown in Table 5.

TABLE 5
Effect of 0 and 2.0 ASPU/gds OPTIMAX ® L-1000 dose at constant
OPTIMALT ® BBA and SPEZYME ® XTRA dose on the solubilization
of granular wheat starch at pH 5.0 and 55° C. and the resulting
sugar composition.

| OPTIMAX ® | | | | | | |
|---|---|---|---|---|---|---|
| L-1000 | Time | Solubilization | DP1 | DP2 | DP3 | DP3+ |
| ASPU/gds | hours | % | % | % | % | % |
| 0 | 3 | 51.8 | 0.7 | 58.0 | 1.8 | 39.5 |
| | 24 | 70.3 | 0.8 | 64.6 | 4.1 | 30.6 |
| | 30 | 72.1 | 0.8 | 65.2 | 4.4 | 29.6 |
| | 48 | 75.3 | 0.8 | 66.1 | 5.1 | 27.9 |
| 2 | 3 | 46.6 | 1.0 | 82.3 | 7.0 | 9.7 |
| | 24 | 69.0 | 1.1 | 84.9 | 9.2 | 4.8 |
| | 30 | 71.3 | 1.1 | 84.8 | 9.3 | 4.9 |
| | 48 | 75.2 | 1.1 | 83.9 | 9.5 | 5.5 |

Table 5 shows that the solubilization of granular starch under the conditions of the experiment was not affected by the addition of the pullulanase OPTIMAX® L-1000. The sugar composition was significantly affected by the presence of pullulanase. At 48 hours hydrolysis, the DP2 increases from 66.1% to 83.9%, DP3 increases from 5.1% to 9.5% and DP3+ decreases from 27.9% to 5.5% due to the presence of 2.0 ASPU/gds OPTIMAX® L-1000. The DP1 is only marginally affected by the addition of pullulanase.

This experiment shows that the presence of a debranching enzyme is required for the production of a hydrolysate with a maltose content of more than 70%.

### Example 6

In this experiment several different alpha-amylases were tested to demonstrate that ultra high maltose syrups can be obtained, independent of the type of alpha-amylase. Different alpha-amylases were added to the hydrolysis of granular wheat starch (Roquette wheat bag starch) at pH 5.0 and 55° C. in the presence of OPTIMALT® BBA and OPTIMAX® L-1000.

A 32% DS aqueous slurry of wheat starch was incubated at pH 5.0 and 55° C. with 2.52 DP°/gds OPTIMALT® BBA, 2.0 ASPU/gds OPTIMAX L-1000 and an alpha-amylase for 48 hours. The following alpha-amylases were tested: SPEZYME® XTRA at 0.1 AAU/gds, SPEZYME® FRED at 0.261 LU/gds, SPEZYME® LT 300 at 0.15 RAU/gds, BAN® 480 L at 0.002 KNU/gds, Liquozyme® Supra at 0.001 KNU/gds, Liquozyme® SCDS at 0.0022 KNU/gds, CLARASE® L at 4.1 SKBU/gds and MAX-LIFE™ P100 from DuPont-Danisco at 0.88 MAA/gds.

The slurry was constantly stirred and samples were taken at different time intervals for determination of % starch solubilization and sugar composition. The solubilization and sugar composition results are shown in Table 6.

TABLE 6
Percentage starch solubilization of granular wheat starch at pH 5.0 and 55° C. and the
resulting sugar composition from hydrolysis of granular starch with 2.52 DP°/gds
OPTIMALT ® BBA, 2.0 ASPU/gds OPTIMAX ® L-1000 and various alpha amylases.

| | | OPTIMALT ® | OPTIMAX ® | | | | | | |
|---|---|---|---|---|---|---|---|---|---|
| Alpha | Dose | BBA | L-1000 | Time | Solubilization | DP1 | DP2 | DP3 | DP3+ |
| amylase | units/gds | DP°/gds | ASPU/gds | hours | % | % | % | % | % |
| SPEZYME ® | 0.1 AAU | 2.52 | 2 | 2.75 | 39.7 | 1.0 | 83. | 7.2 | 7.8 |
| | | | | 18.25 | 62.6 | 1.0 | 84. | 9.2 | 5.5 |
| | | | | 25 | 66.0 | 1.0 | 84. | 9.4 | 5.6 |
| | | | | 42.25 | 70.8 | 1.0 | 80. | 9.7 | 8.4 |
| | | | | 48 | 71.9 | 1.0 | 80. | 9.8 | 9.2 |
| SPEZYME ® | 0.261 LU | 2.52 | 2 | 2.75 | 32.9 | 1.1 | 86. | 7.1 | 5.7 |
| | | | | 18.25 | 53.9 | 1.1 | 85. | 9.2 | 4.2 |
| | | | | 25 | 57.0 | 1.1 | 84. | 9.5 | 4.5 |
| | | | | 42.25 | 60.6 | 1.1 | 81. | 10.1 | 7.5 |
| | | | | 48 | 61.5 | 1.1 | 80. | 10.2 | 8.4 |
| SPEZYME ® LT | 0.15 RAU | 2.52 | 2 | 2.75 | 35.6 | 1.1 | 86. | 6.7 | 5.9 |
| | | | | 18.25 | 51.3 | 1.0 | 86. | 8.0 | 4.9 |
| | | | | 25 | 53.5 | 1.1 | 84. | 8.2 | 6.3 |
| | | | | 42.25 | 56.5 | 1.0 | 80. | 8.5 | 9.7 |
| | | | | 48 | 57.3 | 1.1 | 79. | 8.6 | 10.6 |
| BAN ® 480 L | 0.002 | 2.52 | 2 | 2.75 | 29.8 | 1.1 | 87. | 7.0 | 4.8 |
| | | | | 18.25 | 44.8 | 1.1 | 87. | 7.9 | 3.7 |
| | | | | 25 | 47.2 | 1.1 | 86. | 8.0 | 4.0 |
| | | | | 42.25 | 50.3 | 1.1 | 84. | 8.2 | 5.9 |
| | | | | 48 | 51.1 | 1.1 | 83. | 8.3 | 6.9 |
| Liquozyme ® | 0.001 | 2.52 | 2 | 2.75 | 28.8 | 1.1 | 87. | 6.7 | 4.4 |
| | | | | 18.25 | 48.6 | 1.1 | 86. | 8.1 | 4.0 |
| | | | | 25 | 51.4 | 1.1 | 86. | 8.3 | 4.3 |
| | | | | 42.25 | 54.5 | 1.1 | 82. | 8.7 | 7.4 |
| | | | | 48 | 55.3 | 1.1 | 81. | 8.9 | 8.3 |
| Liquozyme ® | 0.0022 | 2.52 | 2 | 2.75 | 35.4 | 1.1 | 85. | 8.1 | 5.7 |
| | | | | 18.25 | 48.9 | 1.0 | 82. | 10.0 | 6.3 |
| | | | | 25 | 50.9 | 1.1 | 80. | 10.3 | 7.7 |
| | | | | 42.25 | 54.4 | 1.0 | 76. | 10.8 | 11.8 |
| | | | | 48 | 55.8 | 1.0 | 74. | 10.9 | 13.3 |
| CLARASE ® L | 4.1 SKBU | 2.52 | 2 | 3.5 | 38.2 | 1.0 | 78. | 15.3 | 5.3 |
| | | | | 20 | 51.1 | 1.2 | 81. | 14.2 | 3.6 |
| | | | | 28 | 49.0 | 1.2 | 81. | 13.8 | 3.9 |
| | | | | 44 | 51.6 | 1.3 | 81. | 13.3 | 4.3 |
| | | | | 48 | 51.3 | 1.3 | 81. | 13.2 | 4.3 |
| MAX- | 0.88 | 2.52 | 2 | 3.5 | 24.9 | 1.2 | 88. | 4.7 | 5.3 |
| | | | | 20 | 41.7 | 2.0 | 90. | 3.5 | 3.9 |
| | | | | 28 | 43.3 | 1.9 | 89. | 3.4 | 4.8 |
| | | | | 44 | 48.2 | 2.2 | 90. | 2.8 | 4.6 |
| | | | | 48 | 48.8 | 2.2 | 89. | 2.8 | 5.1 |

Table 6 shows that independent of the type of alpha-amylase in hydrolysis reactions according to the present teachings resulted in a very high DP2 content (>80%). Variations in sugar compositions between the different hydrolysis reactions with SPEZYME® FRED, SPEZYME® XTRA, SPEZYME® LT 300, BAN® 480 L, Liquozyme® Supra and Liquozyme® SCDS are small. Peak DP2 varies between 84.3% and 87.7%. After 48 hours reaction DP3 varies between 8.3 and 10.9%, DP3+ varies between 6.9% and 13.3% and DP1 is constant at 1.0-1.1%. The largest differences observed are the differences in % solubilization of the granular starch. Highest starch solubilization is obtained with SPEZYME® XTRA, followed by SPEZYME® FRED, SPEZYME® LT 300, Liquozyme® SCDS, Liquozyme® Supra and BAN® 480 L. The differences may be a reflection of different starch solubilization capability or simply an effect of differences in added alpha-amylase activity.

The sugar profiles obtained with the maltogenic alpha-amylase, MAX-LIFETMP100, and the fungal alpha-amylase CLARASE® L differ from the other alpha-amylases, especially in DP3 and DP2. DP2 for CLARASE® L is very similar to the other alpha-amylases but for MAX-LIFE™ P100 it is several percent higher with peak at 90.6%. Bigger differences between these two enzymes and the other alpha-amylases are observed in the DP3 content. After 48 hours reaction the DP3 content is higher for CLARASE® L at 13.2% and lower for MAX-LIFE™ P100 at 2.8% compared to the other alpha-amylases. Solubilization of the granular starch with either of these two enzymes are a little lower at 48.4% and 51.3% than for the other alpha-amylases.

### Example 7

In this experiment several different beta-amylases, maltogenic alpha-amylases and fungal alpha-amylases were tested to see which enzymes can produce ultra high maltose syrups. The different enzymes were added to the hydrolysis of granular wheat starch (Roquette wheat bag starch) at pH 5.0 and 55° C. in the presence of SPEZYME® XTRA and OPTIMAX® L-1000.

A 32% DS aqueous slurry of wheat starch was incubated at pH 5.0 and 55° C. with 0.1 AAU/gds SPEZYME®XTRA, 2.0 ASPU/gds OPTIMAX® L-1000 and a beta-amylase, maltogenic alpha-amylase or fungal alpha-amylase for 48 hours. The following enzymes were tested: OPTIMALT® BBA at 2.52 DP°/gds, β-amylase#1500S (SBA) at 2.52 DP °/gds, Wheat beta-amylase (WBA) at 2.52 DP °/gds, Betalase 1500L at 2.52 DP°/gds, Maltogenase L at 2.0 MANU/gds, MAX-LIFE™ P100 at 1.76 MAA/gds and CLARASE® L at 41 SKBU/gds

Wheat beta-amylase was obtained through a simple extraction from wheat.

The slurry was constantly stirred and samples were taken at different time intervals for determination of % starch solubilization and sugar composition. The solubilization and sugar composition results are shown in Table 7.

TABLE 7
Percentage starch solubilization of granular wheat starch at pH 5.0 and 55° C. and the
resulting sugar composition from hydrolysis of granular starch with 0.1 AAU/gds
SPEZYME ® XTRA, 2.0 ASPU/gds OPTIMAX ® L-1000 and various beta-amylases,
maltogenic alpha-amylases or fungal alpha-amylases.

| | | SPEZYME ® | OPTIMAX ® | | | | | | |
|---|---|---|---|---|---|---|---|---|---|
| | dose | XTRA | L-1000 | Time | Solubilization | DP1 | DP2 | DP3 | DP3+ |
| Enzyme | Units | AAU/gds | ASPU/gds | hours | % | % | % | % | % |
| WBA | 2.52 DP° | 0.1 | 2 | 3.5 | 46.5 | 1.0 | 81.7 | 6.5 | 10.8 |
| | | | | 20 | 63.1 | 1.1 | 85.5 | 8.3 | 5.1 |
| | | | | 28 | 66.5 | 0.8 | 85.6 | 8.5 | 5.1 |
| | | | | 44 | 71.2 | 1.2 | 84.6 | 8.5 | 5.7 |
| | | | | 48 | 72.1 | 1.2 | 84.4 | 8.6 | 5.8 |
| SBA | 2.52 DP° | 0.1 | 2 | 3.5 | 40.3 | 1.0 | 80.0 | 7.7 | 11.3 |
| | | | | 20 | 61.0 | 1.0 | 82.0 | 10.1 | 6.8 |
| | | | | 28 | 64.4 | 0.9 | 80.8 | 10.5 | 7.7 |
| | | | | 44 | 68.5 | 0.9 | 79.0 | 11.1 | 9.0 |
| | | | | 48 | 69.2 | 1.0 | 78.5 | 11.2 | 9.4 |
| Betalase | 2.52 DP° | 0.1 | 2 | 3.5 | 51.5 | 1.4 | 73.4 | 18.9 | 6.2 |
| | | | | 20 | 66.5 | 2.1 | 72.9 | 20.3 | 4.7 |
| | | | | 28 | 69.2 | 2.3 | 72.6 | 20.1 | 4.9 |
| | | | | 44 | 72.3 | 2.6 | 72.2 | 19.7 | 5.4 |
| | | | | 48 | 73.0 | 2.8 | 72.1 | 19.6 | 5.5 |
| OPTIMALT ® | 2.52 DP° | 0.1 | 2 | 3.5 | 43.0 | 1.0 | 83.1 | 7.3 | 8.6 |
| | | | | 20 | 63.2 | 1.0 | 85.1 | 9.3 | 4.6 |
| | | | | 28 | 66.3 | 1.0 | 84.7 | 9.5 | 4.8 |
| | | | | 44 | 70.3 | 1.0 | 82.7 | 9.8 | 6.5 |
| | | | | 48 | 71.0 | 1.0 | 82.0 | 9.8 | 7.2 |
| MAX-LIFE | 1.76 | 0.1 | 2 | 2.75 | 30.7 | 1.9 | 40.0 | 5.6 | 52.4 |
| | | | | 18.25 | 48.1 | 5.0 | 62.1 | 7.9 | 25.0 |
| | | | | 25 | 52.4 | 5.6 | 64.7 | 7.6 | 22.1 |
| | | | | 42.25 | 59.7 | 6.9 | 69.2 | 6.8 | 17.2 |
| | | | | 48 | 61.7 | 7.1 | 69.8 | 6.5 | 16.6 |
| Maltogenase | 2.0 | 0.1 | 2 | 2.75 | 30.8 | 2.6 | 41.0 | 5.4 | 50.9 |
| | | | | 18.25 | 47.1 | 7.0 | 63.3 | 6.9 | 22.8 |
| | | | | 25 | 51.1 | 7.9 | 65.8 | 6.5 | 19.8 |
| | | | | 42.25 | 57.9 | 9.7 | 69.8 | 5.6 | 14.9 |
| | | | | 48 | 59.9 | 10.1 | 70.5 | 5.4 | 14.1 |
| CLARASE ® | 41 SKBU | 0.1 | 2 | 2.5 | 41.1 | 3.5 | 60.1 | 25.8 | 10.6 |
| | | | | 19 | 56.4 | 6.8 | 65.3 | 20.7 | 7.2 |
| | | | | 26.5 | 59.0 | 7.4 | 64.4 | 20.6 | 7.6 |
| | | | | 42 | 62.6 | 8.1 | 62.6 | 20.6 | 8.7 |
| | | | | 48 | 63.7 | 8.3 | 61.8 | 20.7 | 9.1 |

Table 7 shows that the reactions with beta-amylases from wheat (WBA), barley (BBA) and soy (SBA) result in similar high DP2 content (>80%). Variations in sugar compositions and solubilization of granular starch are small for the different hydrolysis reactions with these three beta-amylases. Using Betalase 1500L results in a similar solubilization of the granular starch, but a 10-13% lower DP2 content and a 8-11% higher DP3 content. The DP1 content is a little bit higher at 2.8% and the DP3+ is a little lower compared to OPTIMALT® BBA. These differences are likely caused by the presence of alpha-amylase in Betalase 1500L.

With the two maltogenic alpha-amylases, Maltogenase L and MAX-LIFE™ P100 lower solubilities and lower DP2 levels were obtained compared to OPTIMALT® BBA. Solubilization for both maltogenic alpha-amylases was very similar at 59.9-61.7%. In contrast to Betalase 1500, the DP3 level for maltogenic alpha-amylases is lower than from OPTIMALT® BBA, but the DP1 at 7.1 and 10.1% and DP3+ at 16.6 and 14.1% are much higher than obtained with OPTIMALT® BBA. The low DP3 and high DP1 content is likely due to the ability of these maltogenic alpha amylases to degrade DP3 into DP2 and DP1. The high DP3+ content indicates that the maltogenic alpha amylase action results in starch fragments that cannot, or only very slowly, be degraded by the present enzymes.

With the fungal alpha-amylase CLARASE® L, the lowest DP2 content is formed among all the maltogenic enzymes. The maximum DP2 content is 65.3% for CLARASE® L. The solubilization obtained with CLARASE® L is at 63.7%, similar to that of the two maltogenic alpha-amylases but behind that of the beta-amylases. The DP3 content with CLARASE® L is the highest of all reactions in example 7.

This example shows that any of a variety of maltogenic enzymes can be used to produce a maltose hydrolysate with >60% maltose in the presence of an alpha-amylase and debranching enzyme. This example further shows that any beta-amylase can be used to produce a maltose hydrolysate with >80% maltose in the presence of an alpha-amylase and debranching enzyme.

### Example 8

In this experiment several different debranching enzymes were tested to demonstrate that ultra high maltose syrups can be obtained, independent of the type of debranching enzyme. Different debranching enzymes were added to the hydrolysis of granular wheat starch (Roquette wheat bag starch) at pH 5.0 and 55° C. in the presence of SPEZYME® XTRA and OPTIMALT® BBA.

A 32% DS aqueous slurry of wheat starch was incubated at pH 5.0 and 55° C. with 2.52 DP°/gds OPTIMALT® BBA, 0.1 AAU/gds SPEZYME® XTRA and a debranching enzyme for 48 hours. The following debranching enzymes were tested: OPTIMAX® L-1000 at 2.0 ASPU/gds, Promozyme® D2 at 2.0 KNUN/gds and ISOAMYLASE from Pseudomonas sp from Megazyme International (ISOAMYLASE) at 0.89 U/gds.

The slurry was constantly stirred and samples were taken at different time intervals for determination of % starch solubilization and sugar composition. The solubilization and sugar composition results are shown in Table 8.

TABLE 8
Percentage starch solubilization of granular wheat starch at pH 5.0 and 55° C. and the
resulting sugar composition from hydrolysis of granular starch with 2.52 DP°/gds
OPTIMALT BBA, 0.1 AAU/gds SPEZYME ® XTRA and various debranching enzymes.

| | dose | SPEZYME ® | OPTIMALT ® | | | | | | |
|---|---|---|---|---|---|---|---|---|---|
| | Units/ | XTRA | BBA | Time | Solubilization | DP1 | DP2 | DP3 | DP3+ |
| Enzyme | gds | AAU/gds | DP°/gds | hours | % | % | % | % | % |
| OPTIMAX ® L- | 2 ASPU | 0.1 | 2.52 | 3.5 | 43.0 | 1.0 | 83.1 | 7.3 | 8.6 |
| 1000 | | | | | | | | | |
| | | | | 20 | 63.2 | 1.0 | 85.1 | 9.3 | 4.6 |
| | | | | 28 | 66.3 | 1.0 | 84.7 | 9.5 | 4.8 |
| | | | | 44 | 70.3 | 1.0 | 82.7 | 9.8 | 6.5 |
| | | | | 48 | 71.0 | 1.0 | 82.0 | 9.8 | 7.2 |
| ISOAMYLASE | 0.89 U | 0.1 | 2.52 | 2.5 | 29.0 | 0.9 | 80.0 | 8.0 | 11.1 |
| | | | | 19 | 47.3 | 0.9 | 79.1 | 10.5 | 9.5 |
| | | | | 26.5 | 50.6 | 0.9 | 78.8 | 10.9 | 9.4 |
| | | | | 42 | 55.2 | 0.9 | 76.6 | 11.4 | 11.1 |
| | | | | 48 | 56.6 | 0.9 | 75.8 | 11.5 | 11.8 |
| Promozyme ® | 2 PNUN | 0.1 | 2.52 | 3.5 | 43.9 | 0.9 | 79.1 | 6.7 | 13.3 |
| D2 | | | | | | | | | |
| | | | | 20 | 61.5 | 1.0 | 82.5 | 9.5 | 7.1 |
| | | | | 28 | 64.2 | 1.0 | 81.0 | 9.8 | 8.3 |
| | | | | 44 | 67.8 | 1.0 | 77.8 | 10.2 | 11.0 |
| | | | | 48 | 68.5 | 1.0 | 77.2 | 10.3 | 11.5 |

Table 8 shows that with both pullulanases, OPTIMAX® L-1000 and Promozyme® D2 a very similar and high DP2 content (>80%) can be reached. Variations in sugar compositions between the different hydrolysis reactions are small. Peak DP2 is 85.1% for OPTIMAX® L-1000 and 82.5% for Promozyme® D2. After 48 hours reaction, DP3 content is nearly identical for these two pullulanases, DP3+ is higher for Promozyme® D2 at 11.5% than for OPTIMAX® L-1000 at 7.2% and DP1 is constant at 0.9-1.0% for both pullulanases. There is also hardly any differences in % solubilization of the granular starch for both pullulanases. With OPTIMAX® L-1000 a slightly higher solubilization is obtained (71%) than with Promozyme® D2 (68.5%).

For the ISOAMYLASE a very similar profile is seen as with the pullulanases. DP2 levels are a little lower with peak DP2 at 80% and final DP2 at 48 hours of 75.8%. DP3 and DP3+ levels are similar to those obtained with Promozyme® D2 and are a little higher than obtained with OPTIMAX® L-1000. The solubilization is much lower with the ISOAMYLASE than with the 2 pullulanases.

This example shows that a high maltose hydrolysate with >75% maltose can be obtained with the current teachings independent of the type of debranching enzyme used.

### Example 9

In this experiment the effect of adding a maltogenic amylase and/or a fungal alpha-amylase in addition to SPEZYME® XTRA, OPTIMALT® BBA and OPTIMAX® L-1000 was studied in the hydrolysis of granular wheat starch (Roquette wheat bag starch).

A 32% DS aqueous slurry of wheat starch was incubated at pH 5.0 and 55° C. with 2.52 DP°/gds OPTIMALT® BBA, 0.1 AAU/gds SPEZYME® XTRA and 2.0 ASPU/gds OPTIMAX® L-1000 for 48 hours. In the first set of experiments either MAX-LIFE™ P100, CLARASE® L or a blend of MAX-LIFE™ P100 and CLARASE® L were added at the start of the reaction. The dosages used are shown in Table 9a. In the second set of experiments either MAX-LIFE™ P100, CLARASE® L or a blend of MAX-LIFE™ P100 and CLARASE® L were added 24 hours after the start of the reaction, also referred to as staging. The dosages used are shown in Table 9b.

The slurry was constantly stirred and samples were taken at different time intervals for determination of % starch solubilization and sugar composition. The solubilization and sugar composition results are shown in Table 9a and 9b.

TABLE 9A
Percentage starch solubilization of granular wheat starch at pH 5.0 and 55° C.
and the resulting sugar composition from hydrolysis of granular starch with 2.52
DP°/gds OPTIMALT ® BBA, 0.1 AAU/gds SPEZYME ® XTRA and 2.0 ASPU/gds
OPTIMAX ® L-1000 where additional CLARASE ® L and/or MAX-LIFE ™ P100 was
added at the start of the hydrolysis.

| | MAX- | | | | | | |
|---|---|---|---|---|---|---|---|
| | LIFE ™ | | | | | | |
| CLARASE ® L | P100 | Time | Solubilization | DP1 | DP2 | DP3 | DP3+ |
| SKBU/gds | MAA/gds | hours | % | % | % | % | % |
| | | 3.5 | 44.0 | 1.0 | 83.8 | 7.4 | 7.8 |
| | | 20 | 63.0 | 1.0 | 84.7 | 9.3 | 5.0 |
| | | 28 | 65.5 | 1.2 | 83.3 | 9.6 | 5.9 |
| | | 44 | 69.0 | 1.0 | 81.9 | 9.8 | 7.3 |
| | | 48 | 69.5 | 1.0 | 81.4 | 9.8 | 7.7 |
| | 1.76 | 3.5 | 42.2 | 1.1 | 84.0 | 6.5 | 8.5 |
| | | 20 | 62.3 | 2.0 | 87.1 | 6.2 | 4.8 |
| | | 28 | 65.2 | 2.2 | 86.8 | 5.7 | 5.3 |
| | | 44 | 68.9 | 2.7 | 85.2 | 4.7 | 7.4 |
| | | 48 | 69.4 | 2.8 | 84.8 | 4.5 | 8.0 |
| 2 | | 3.5 | 48.6 | 1.6 | 71.5 | 20.5 | 6.4 |
| | | 20 | 63.5 | 2.5 | 75.0 | 17.8 | 4.7 |
| | | 28 | 65.4 | 2.7 | 74.8 | 17.3 | 5.2 |
| | | 44 | 69.1 | 3.0 | 74.5 | 16.7 | 5.7 |
| | | 48 | 69.8 | 3.0 | 74.4 | 16.8 | 5.8 |
| 2 | 3.52 | 2.5 | 39.7 | 3.8 | 72.0 | 17.3 | 6.9 |
| | | 19 | 56.9 | 8.3 | 78.3 | 8.3 | 5.1 |
| | | 26.5 | 60.1 | 9.1 | 79.0 | 6.6 | 5.2 |
| | | 42 | 64.7 | 10.0 | 80.0 | 4.5 | 5.4 |
| | | 48 | 65.9 | 10.2 | 80.0 | 4.0 | 5.8 |

TABLE 9B
Percentage starch solubilization of granular wheat starch at pH 5.0 and 55° C.
and the resulting sugar composition from hydrolysis of granular starch with 2.52
DP°/gds OPTIMALT ® BBA, 0.1 AAU/gds SPEZYME ® XTRA and 2.0 ASPU/gds
OPTIMAX ® L-1000, where additional CLARASE ® L and/or MAX-LIFE ™ P100
was added 24 hours after the start of the hydrolysis.

| | MAX- | | | | | | |
|---|---|---|---|---|---|---|---|
| | LIFE ™ | | | | | | |
| CLARASE ® L | P100 | Time | Solubilization | DP1 | DP2 | DP3 | DP3+ |
| SKBU/gds | MAA/gds | hours | % | % | % | % | % |
| | | 2.5 | 34.9 | 1.0 | 84.2 | 7.4 | 7.4 |
| | | 19 | 56.8 | 1.1 | 85.1 | 9.5 | 4.3 |
| | | 26.5 | 60.7 | 1.1 | 84.5 | 9.8 | 4.7 |
| | | 42 | 65.2 | 1.1 | 82.9 | 10.0 | 6.0 |
| | | 48 | 66.3 | 1.1 | 82.2 | 10.1 | 6.6 |
| | 3.52 | 2.5 | 34.1 | 1.1 | 83.9 | 7.5 | 7.5 |
| | | 19 | 53.5 | 1.0 | 82.0 | 9.9 | 7.1 |
| | | 26.5 | 55.6 | 1.6 | 81.0 | 8.9 | 8.5 |
| | | 42 | 59.1 | 3.6 | 83.2 | 4.5 | 8.6 |
| | | 48 | 60.3 | 4.1 | 83.8 | 3.8 | 8.4 |
| 2 | | 2.5 | 34.2 | 1.0 | 83.8 | 7.5 | 7.7 |
| | | 19 | 53.1 | 1.0 | 79.7 | 9.8 | 9.5 |
| | | 26.5 | 57.7 | 1.8 | 77.5 | 15.4 | 5.3 |
| | | 42 | 63.7 | 3.8 | 74.9 | 15.6 | 5.6 |
| | | 48 | 65.2 | 4.2 | 74.0 | 15.8 | 6.1 |
| 2 | 3.52 | 2.5 | 34.6 | 1.1 | 84.1 | 7.5 | 7.3 |
| | | 19 | 53.7 | 1.0 | 81.6 | 9.8 | 7.5 |
| | | 26.5 | 58.2 | 2.4 | 80.2 | 12.2 | 5.2 |
| | | 42 | 64.5 | 6.6 | 78.9 | 8.6 | 5.9 |
| | | 48 | 65.9 | 7.6 | 78.3 | 7.8 | 6.4 |

In Table 9a it is shown that the addition of MAX-LIFE™ P100 at the start of the hydrolysis results in higher DP2 levels at 48 hours reaction than when no additional maltogenic enzyme is added. The additional MAX-LIFE™ P100 also resulted in a much lower DP3 content and a slightly higher DP1 content. When CLARASE® L is added at the start of the reaction this results in a much lower DP2 content, a little lower DP3+ content, a little higher DP1 content but a much higher DP3 content.

Addition of both MAX-LIFE™ P100 and CLARASE® L at the start of the hydrolysis reaction results in a final DP2 content at 48 hours of 80%, 1.4% lower than the reaction without additional maltogenic enzymes. At the same time, the DP3+ and DP3 content are much lower when both maltogenic enzymes are added. This can be a significant benefit when the final hydrolysate is further purified with some form of chromatography, since it is easier to separate DP1 and DP2 than DP2 and DP3.

From Table 9b, it can be seen that there does not seem to be a benefit from staging the addition of the extra maltogenic enzymes.

### Example 10

In this experiment granular starch was treated with a low alpha-amylase dose and a high beta-amylase dose in the presence of a debranching enzyme on corn and tapioca starch at pH 5.0 and 60-68° C.

A 32% DS aqueous slurry of corn starch (Cargill Gel™ 3240 ™ unmodified dry corn starch) or tapioca starch (National Starch, Corn Products, unmodified starch) was incubated at pH 5.0 and 60° C. with 0.1 AAU/gds SPEZYME® XTRA, 2.0 ASPU/gds OPTIMAX® L-1000 and 2.52 DP°/gds OPTIMALT® BBA for 48 hours.

A 34% DS aqueous slurry of corn starch (Cargill Gel™ 3240 ™ unmodified dry corn starch) or tapioca starch (National Starch, Corn Products, unmodified starch) was incubated at pH 5.0 and 68° C. with 0.1 AAU/gds SPEZYME® XTRA, 9.7 ASPU/gds thermostable pullulanase from Fervidobacterium nodosum and 1.9 DP°/gds β-amylase#1500S (SBA) for 48 hours.

The slurry was constantly stirred and samples were taken at different time intervals for determination of % starch solubilization and sugar composition. The enzyme dose and results are shown in Table 10.

TABLE 10
Solubilization of granular starch from tapioca and corn at 32-34% DS, pH 5.0 and 60-68° C. and the resulting sugar
composition

| | | OPTIMALT ® | | OPTIMAX ® | | | | | | | |
|---|---|---|---|---|---|---|---|---|---|---|---|
| Raw | Temp | BBA | SBA | L-1000 | FnoPul | Time | Solub. | DP1 | DP2 | DP3 | DP3+ |
| Material | ° C. | DP°/gds | DP°/gds | ASPU/gds | ASPU/gds | hours | % | % | % | % | % |
| Corn | 60 | 2.52 | | 2 | | 16 | 28.9 | 0.6 | 75.2 | 16.9 | 7.3 |
| (32% DS) | | | | | | 24 | 32.8 | 0.6 | 74.2 | 17.5 | 7.7 |
| | | | | | | 40 | 38.3 | 0.6 | 72.0 | 18.5 | 8.8 |
| | | | | | | 48 | 40.5 | 0.7 | 71.2 | 18.9 | 9.3 |
| Tapioca | 60 | 2.52 | | 2 | | 16 | 40.0 | 0.2 | 81.4 | 12.9 | 5.5 |
| (32% DS) | | | | | | 24 | 43.8 | 0.3 | 81.8 | 12.7 | 5.2 |
| | | | | | | 40 | 49.1 | 0.3 | 81.1 | 13.1 | 5.5 |
| | | | | | | 48 | 52.9 | 0.3 | 80.3 | 13.4 | 6.0 |
| Corn | 68 | | 1.9 | | 9.7 | 6 | 51.6 | 0.0 | 82.5 | 10.1 | 7.4 |
| (34% DS) | | | | | | 24 | 61.2 | 0.0 | 74.1 | 11.6 | 14.3 |
| | | | | | | 48 | 63.6 | 0.4 | 70.9 | 13.2 | 15.5 |
| Tapioca | 68 | | 1.9 | | 9.7 | 18 | 73.6 | 0.0 | 81.5 | 9.6 | 8.9 |
| (34% DS) | | | | | | 24 | 76.2 | 0.0 | 81.2 | 9.8 | 9.0 |
| | | | | | | 48 | 69.7 | 0.0 | 77.8 | 10.0 | 12.1 |

From Table 10 it is clear that at 60° C. the solubilization of corn and tapioca starch is much lower than it is for wheat starch at the same temperature (see Examples 1-3). The lower solubilization is likely a result of the higher gelatinization temperature for corn and tapioca. At 68° C., the solubilization of both corn and tapioca starch is much higher than at 60° C. At the higher temperature more thermostable enzymes were used due to the lower stability of OPTIMALT® BBA and OPTIMAX® L-1000 at this elevated temperature. For corn at 60 and 68° C., the DP2 content after 48 hours reaction is 70.9-71.2%. For tapioca, the DP2 content after 48 hours reaction is 77.8-80.3% for the two temperatures. DP1 is very low for both raw materials and the two temperatures. There is more difference in DP3 and DP3+. At 68° C., the DP3 content is lower for both corn and tapioca than at 60° C. whereas the DP3+ content is higher.

This experiment shows that with the current invention a >70% maltose syrup can be produced from corn starch and >80% maltose syrup from tapioca starch. Elevated temperatures seem to mainly influence the % solubilization and to a much smaller extend the maltose content, as was seen for wheat starch as well (see Example 12).

### Example 11

In this experiment the concept of treating granular starch with a low alpha-amylase dose and a high beta-amylase dose in the presence of a debranching enzyme was tested on whole ground wheat and whole ground barley at pH 5.0 and 55° C. Both whole ground wheat and barley contain substantial amounts of endogenous beta-amylase. In this experiment, tests were done making use of the endogenous beta-amylase activity as well as adding additional beta-amylase.

A 32% DS aqueous slurry of whole ground wheat (Arie Blok Graanproducten, Woerden, The Netherlands) or whole ground barley (Arie Blok Graanproducten, Woerden, The Netherlands) was incubated at pH 5.0 and 55° C. with 0.1 AAU/gds SPEZYME® XTRA, 1.0 ASPU/gds OPTIMAX® L-1000 and 0 or 2.52 DP °/gds OPTIMALT® BBA for 48 hours. In order to prevent high viscosities caused by solubilized fibers from the whole ground grains, 2.3 CMCU/gds of OPTIMASH™ BG was added to each reaction. The slurry was constantly stirred and samples were taken at different time intervals for determination of % starch solubilization and sugar composition. The enzyme dose and results are shown in Table 11.

TABLE 11
Solubilization of granular starch from whole ground wheat at pH 5.0 and 55° C. and the
resulting sugar composition.

| | SPEZYME ® | OPTIMALT ® | OPTIMAX ® | | | | | | |
|---|---|---|---|---|---|---|---|---|---|
| | XTRA | BBA | L-1000 | Time | Solubilization | DP1 | DP2 | DP3 | DP3+ |
| Grain | AAU/g DS | DP°/gds | ASPU/gds | hours | % | % | % | % | % |
| Wheat | 0.1 | | 2 | 2.75 | 34.3 | 14.4 | 44.7 | 11.0 | 22.5 |
| | | | | 18.25 | 55.9 | 15.6 | 48.8 | 15.7 | 15.1 |
| | | | | 27.25 | 62.1 | 15.4 | 48.2 | 17.0 | 14.3 |
| | | | | 42.5 | 66.1 | 15.3 | 45.9 | 18.5 | 14.6 |
| | | | | 48 | 66.6 | 15.4 | 44.9 | 18.6 | 15.5 |
| Wheat | 0.5 | | 2 | 2.75 | 40.6 | 11.1 | 51.3 | 12.6 | 20.0 |
| | | | | 18.25 | 64.4 | 13.8 | 52.0 | 16.4 | 13.9 |
| | | | | 27.25 | 69.7 | 14.1 | 50.5 | 17.6 | 13.7 |
| | | | | 42.5 | 75.0 | 14.9 | 47.9 | 19.2 | 14.0 |
| | | | | 48 | 75.3 | 14.3 | 47.6 | 19.7 | 14.4 |
| Wheat | 2 | | 2 | 2.75 | 49.2 | 9.1 | 55.5 | 13.6 | 17.9 |
| | | | | 18.25 | 72.7 | 12.9 | 54.3 | 16.3 | 13.2 |
| | | | | 27.25 | 79.5 | 14.6 | 52.0 | 17.2 | 12.9 |
| | | | | 42.5 | 85.4 | 15.5 | 49.3 | 18.7 | 13.1 |
| | | | | 48 | 85.4 | 15.3 | 48.5 | 19.3 | 13.6 |
| Wheat | 2 | 2.52 | 2 | 2.75 | 48.5 | 9.3 | 55.3 | 13.5 | 17.9 |
| | | | | 18.25 | 72.0 | 13.1 | 54.5 | 16.2 | 12.9 |
| | | | | 27.25 | 78.1 | 14.9 | 52.0 | 16.8 | 13.0 |
| | | | | 42.5 | 86.8 | 16.8 | 49.0 | 18.0 | 13.0 |
| | | | | 48 | 86.7 | 16.0 | 48.9 | 18.7 | 13.1 |
| Barley | 0.1 | | 2 | 2.75 | 29.0 | 17.3 | 31.2 | 8.8 | 33.8 |
| | | | | 18.25 | 49.1 | 16.7 | 47.1 | 15.5 | 15.9 |
| | | | | 27.25 | 52.0 | 13.8 | 49.2 | 17.2 | 15.3 |
| | | | | 42.5 | 53.1 | 14.1 | 47.8 | 18.7 | 14.6 |
| | | | | 48 | 54.6 | 14.0 | 46.7 | 19.0 | 15.8 |
| Barley | 0.5 | | 2 | 2.75 | 39.1 | 12.5 | 44.0 | 12.5 | 25.7 |
| | | | | 18.25 | 64.0 | 13.1 | 53.9 | 16.3 | 13.2 |
| | | | | 27.25 | 67.7 | 11.1 | 56.1 | 17.5 | 12.3 |
| | | | | 42.5 | 69.4 | 11.3 | 54.7 | 18.7 | 12.3 |
| | | | | 48 | 70.1 | 11.5 | 53.6 | 19.1 | 12.9 |
| Barley | 2 | | 2 | 2.75 | 52.6 | 8.9 | 51.6 | 15.1 | 20.5 |
| | | | | 18.25 | 74.6 | 11.3 | 56.3 | 16.9 | 12.5 |
| | | | | 27.25 | 78.4 | 10.4 | 58.2 | 17.4 | 11.4 |
| | | | | 42.5 | 79.9 | 10.2 | 56.9 | 18.6 | 11.6 |
| | | | | 48 | 80.3 | 10.4 | 56.1 | 18.8 | 12.0 |
| Barley | 2 | 2.52 | 2 | 2.75 | 49.1 | 9.7 | 49.5 | 14.4 | 22.2 |
| | | | | 18.25 | 72.1 | 11.7 | 55.5 | 16.8 | 12.9 |
| | | | | 27.25 | 75.2 | 10.3 | 56.8 | 18.0 | 12.1 |
| | | | | 42.5 | 77.0 | 10.5 | 55.1 | 19.3 | 12.2 |
| | | | | 48 | 77.7 | 10.7 | 54.3 | 19.6 | 12.7 |

Comparing Table 11 with previous tables shows that here are clear differences between reacting whole ground wheat and wheat starch with a low alpha-amylase dose and a high beta-amylase dose in the presence of a debranching enzyme. Solubilization for whole ground wheat and barley are higher than seen for wheat starch by about 10-15%. Solubilization of whole ground wheat is higher than for whole ground barley.

Another difference between the reactions with whole ground grains and their starches is the sugar composition. The DP2 content for whole ground grains is significantly lower than for their starches. For whole ground barley the maximum DP2 is 56.9% and for whole ground wheat the maximum DP2 is 55.5%. These are roughly 30% lower than what can be obtained for wheat starch under similar conditions. Also the DP1, DP3 and DP3+ content is much higher for reactions with the whole ground grains than for reactions with their starches. These differences are likely caused by the presence of other oligosaccharides and carbohydrates from the whole ground grains. These will influence the results presented in Table 11.

In contrast to reactions with wheat starch, where an increasing alpha-amylase dose results in a decrease in DP2, we see the opposite with whole ground wheat. With increasing alpha-amylase dose from 0.1-2.0 AAU/gds the DP2 content increases. This is seen for both whole ground wheat and barley. Adding additional beta-amylase in reactions with 2.0 AAU/gds SPEZYME® XTRA does not increase the DP2 content for either of the whole ground grains. This shows that the amount of endogenous beta-amylase in these whole grain samples is very high and that the ratio of beta-amylase/alpha-amylase dose is also very high. This suggests that the alpha-amylase dose can be further increased to gain solubilization and DP2.

### Example 12

In this experiment the influence of temperature on the solubilization of granular wheat starch (Roquette wheat bag starch) in the presence of SPEZYME® XTRA, OPTIMALT® BBA and OPTIMAX® L-1000, as well as the influence on resulting sugar composition of the hydrolysate was studied.

A 32% DS aqueous slurry of wheat starch was incubated at pH 5.0 and temperatures between 50 and 65° C. with 2.52 DP°/gds OPTIMALT® BBA, 2.0 ASPU/gds OPTIMAX® L-1000 and 0.1 AAU/gds SPEZYME® XTRA for 48 hours. The slurry was constantly stirred and samples were taken at different time intervals for determination of % starch solubilization and sugar composition. The results are shown in Table 12.

TABLE 12
Effect of temperature, at constant SPEZYME ® XTRA, OPTIMALT ®
BBA and OPTIMAX ® L-1000 dose on the solubilization of granular
wheat starch at pH 5.0 and the resulting sugar composition.

| Temp. | Time | Solubilization | DP1 | DP2 | DP3 | DP3+ |
|---|---|---|---|---|---|---|
| ° C. | hours | % | % | % | % | % |
| 50 | 3 | 18.9 | 1.2 | 85.0 | 10.9 | 3.0 |
| | 20 | 39.2 | 1.1 | 82.5 | 11.9 | 4.5 |
| | 27 | 43.3 | 1.3 | 82.8 | 12.1 | 3.8 |
| | 44 | 50.6 | 1.1 | 82.3 | 12.4 | 4.2 |
| | 48 | 51.8 | 1.2 | 82.1 | 12.3 | 4.4 |
| 53 | 4.5 | 34.4 | 1.1 | 85.2 | 8.9 | 4.9 |
| | 22.25 | 50.4 | 1.0 | 83.4 | 10.6 | 5.0 |
| | 27.5 | 52.2 | 1.0 | 82.8 | 10.8 | 5.3 |
| | 47 | 56.8 | 1.0 | 81.5 | 11.4 | 6.0 |
| | 52 | 57.6 | 1.0 | 81.4 | 11.5 | 6.1 |
| 55 | 3 | 44.7 | 1.0 | 83.1 | 7.4 | 8.5 |
| | 20 | 63.2 | 1.0 | 84.9 | 9.5 | 4.6 |
| | 27 | 65.6 | 1.0 | 84.5 | 9.7 | 4.8 |
| | 44 | 69.0 | 0.9 | 82.9 | 10.1 | 6.1 |
| | 48 | 69.6 | 0.9 | 82.7 | 10.1 | 6.2 |
| 57.5 | 3 | 58.6 | 1.1 | 79.0 | 6.4 | 13.4 |
| | 20 | 75.9 | 1.0 | 85.1 | 8.8 | 5.1 |
| | 27 | 78.0 | 1.2 | 84.8 | 9.0 | 5.0 |
| | 44 | 81.5 | 0.9 | 83.8 | 9.3 | 6.0 |
| | 48 | 81.1 | 1.0 | 83.3 | 9.3 | 6.4 |
| 60 | 3 | 72.4 | 0.9 | 76.1 | 5.5 | 17.5 |
| | 20 | 84.4 | 1.0 | 85.3 | 8.3 | 5.4 |
| | 27 | 86.6 | 1.0 | 85.3 | 8.5 | 5.3 |
| | 44 | 89.5 | 0.9 | 84.9 | 8.7 | 5.5 |
| | 48 | 90.0 | 0.9 | 84.7 | 8.7 | 5.6 |
| 65 | 3 | 87.1 | 0.7 | 62.8 | 4.1 | 32.3 |
| | 20 | 90.0 | 0.9 | 71.7 | 7.9 | 19.6 |
| | 27 | 90.4 | 0.8 | 71.6 | 8.5 | 19.1 |
| | 44 | 92.9 | 0.8 | 71.6 | 9.0 | 18.6 |
| | 48 | 93.3 | 0.8 | 71.8 | 9.1 | 18.3 |

From Table 12 it is clear that with increasing temperature, the % solubilization of granular starch increases from 51.8% at 50° C. to 93.3% at 65° C. It is also clear that 65° C. results in much lower DP2 levels, which seems to be related to inactivation of some of the enzymes, mainly pullulanase, judged by the high residual DP3+ content. For temperatures between 50-60° C., the DP2 content is very similar with peak DP2 levels between 85-85.3%. With increasing temperatures the peak DP2 seems to shift towards a later time point and the drop in DP2 content (caused by continued solubilization of starch, not by loss of DP2) is slower at higher temperatures. Differences in DP1 and DP3+ are small between the different temperatures between 50-60° C. For DP3 there is a clear increase with decreasing temperatures. At a low temperature, the solubilization of granular starch is slower than at higher temperatures. This means that the amount of alpha-amylase per gram of dissolved starch is higher at low temperatures than at higher temperatures, resulting in a higher DP3 content. This may indicate that the ratio of alpha-amylase over beta-amylase is especially important at the beginning (e.g. first few hours) of the hydrolysis reaction.

This experiment shows that the maltose content is independent of the temperature of the reaction as long as the temperature is such that the enzymes remain active. A higher temperature does increase solubilization of granular starch.

### Example 13

In this experiment it was studied if there is a benefit for adding four times more beta-amylase and four times more alpha-amylase thereby keeping the ratio of beta-amylase activity over alpha-amylase activity constant. It was also studied if multiple additions of the same beta-amylase over alpha-amylase ratios have an advantage.

A 32% DS aqueous slurry of wheat starch was incubated at pH 5.0 and 55° C. At time zero 2.0 ASPU/gds OPTIMAX® L-1000 was added and either 2.52 DP°/gds OPTIMALT® BBA with 0.1 AAU/gds SPEZYME® XTRA (Test A) or 10.08 DP°/gds OPTIMALT® BBA with 0.4 AAU/gds SPEZYME® XTRA (Test B) were added. To a different reaction (Test C) 2.0 ASPU/gds OPTIMAX® L-1000 was added at time zero together with 2.52 DP°/gds OPTIMALT® BBA and 0.1 AAU/gds SPEZYME® XTRA. At time 7.5, 24 and 30.5 hours again 2.52 DP °/gds OPTIMALT® BBA and 0.1 AAU/gds SPEZYME® XTRA were added. The slurries were constantly stirred and samples were taken at different time intervals for determination of % starch solubilization and sugar composition. The results are shown in Table 13.

TABLE 13
Effect of single, multiple or staged dosing of SPEZYME ® XTRA and OPTIMALT ® BBA
at constant OPTIMAX ® L-1000 dose on the solubilization of granular wheat starch at pH
5.0 and the resulting sugar composition.

| | SPEZYME ® | OPTIMALT ® | | | | | | |
|---|---|---|---|---|---|---|---|---|
| Test | XTRA | BBA | Time | Solubilization | DP1 | DP2 | DP3 | DP3+ |
| | AAU/g DS | DP°/gds | hours | % | % | % | % | % |
| Test A | 0.1 | 2.52 | 3 | 46.6 | 1.0 | 82.3 | 7.0 | 9.7 |
| | | | 24 | 69.0 | 1.1 | 84.9 | 9.2 | 4.8 |
| | | | 30 | 71.3 | 1.1 | 84.8 | 9.3 | 4.9 |
| | | | 48 | 75.2 | 1.1 | 83.9 | 9.5 | 5.5 |
| Test B | 0.4 | 10.08 | 3 | 55.4 | 1.0 | 81.6 | 9.1 | 8.3 |
| | | | 24 | 72.6 | 1.1 | 83.0 | 10.8 | 5.0 |
| | | | 30 | 74.0 | 1.2 | 82.8 | 10.8 | 5.2 |
| | | | 48 | 77.3 | 1.2 | 82.1 | 10.9 | 5.8 |
| Test C | 4 × 0.1 | 4 × 2.52 | 3 | 46.1 | 1.0 | 82.0 | 7.0 | 9.9 |
| | | | 24 | 70.5 | 1.1 | 85.1 | 9.0 | 4.8 |
| | | | 30 | 73.5 | 1.6 | 84.3 | 9.1 | 5.1 |
| | | | 48 | 79.7 | 1.3 | 83.9 | 9.3 | 5.5 |

Adding four times more SPEZYME® XTRA and OPTIMALT® BBA from the start results in faster solubilization of the granular starch than adding a single dose. When the additional SPEZYME® XTRA and OPTIMALT® BBA addition is staged, the initial solubilization of the granular starch (at t=3 hours) is the same as when a single dose is added. This is expected because an equal amount of SPEZYME® XTRA and OPTIMALT® BBA are present at the start of the reaction. Upon additional staged additions of SPEZYME® XTRA and OPTIMALT® BBA, the solubilization exceeds that of both test A and test B, showing that adding alpha-amylase and beta-amylase during the reaction is beneficial. This can be seen as adding more alpha-amylase and beta-amylase as more substrate (solubilized starch) becomes available.

Comparing Test A and Test C shows very similar sugar profiles during the hydrolysis of the granular wheat starch. Test B does show some differences in DP2 and DP3. When a four times higher SPEZYME® XTRA and OPTIMALT® BBA dose is added from the start (Test B), the peak DP2 content (83%) is lower than when a single dose is added (Test A) or when the four times higher dose is staged (Test C). In both Test A and Test C the peak DP2 values are very similar at 84.9% and 85.1%, respectively. A similar trend is seen for DP3, where Test A and Test C have very similar DP3 content but Test B has a higher DP3 content.

### Example 14

This example demonstrates effects of temperature staging (i.e., using more than one temperature to carry out the reaction) on various parameters. The experiments were carried out as described in previous examples with different temperatures and differing time periods as indicated below. These experiments were done in order to improve the process of maltose production from granular starch. In the previous experiments, it was observed that the maltose syrup could be difficult to separate from the insoluble residual material. When the final reaction mixture is centrifuged, three distinct layers can be seen. Such a spin test can be indicative of how easily solids can or cannot be separated from syrup. The three different layers are: bottom layer is residual starch, middle layer is a “gel like” material and the top layer is clear supernatant (maltose syrup). The gel-like layer in the middle makes the separation difficult. The larger the gel-like layer, the more difficult the syrup will be to separate from the residuals. Another factor affecting easiness of separation is the viscosity of the reaction mixture. The lower the viscosity of the final reaction mixture, the better the separation. These experiments were focused on reducing the amount of gel as well as improving the viscosity.

### Effect of Temperature Staging on Viscosity and Filtration

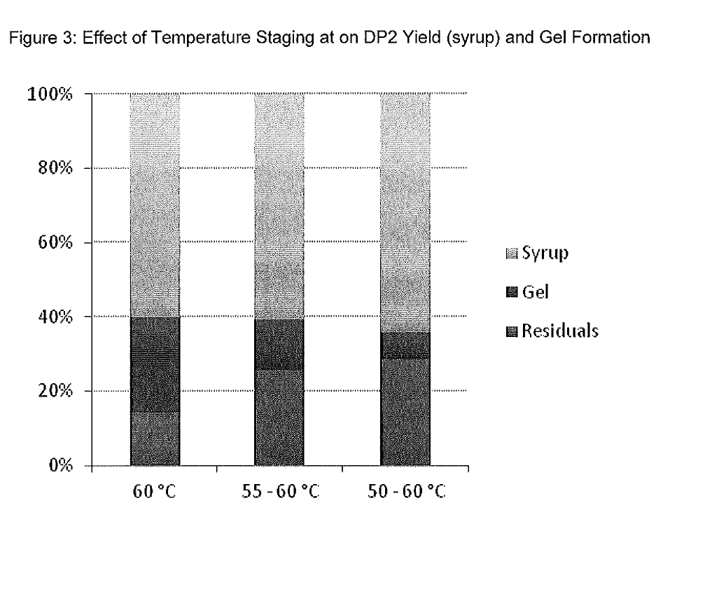

From the previous examples, it is apparent that using a low alpha amylase dosage in combination with a high beta amylase dosage results in syrup with very high maltose. But using a low alpha amylase dosage also results in high viscosity, especially in the initial stage of malto-saccharification. The effect of temperature staging on viscosity and generation of gel layer was measured and it was found that the longer the slurry is kept at 50° C. the lower the viscosity peak as well as lower the gel layer thickness initiated by the heating to 60° C. (FIG. 3 and Table 14 and 15).

TABLE 14

| Time at 50° C. [min] | Peak viscosity [cP] | Peak viscosity [%] |
|---|---|---|
| 0 | 10144 | 100 |
| 10 | 4314 | 43 |
| 20 | 1330 | 13 |
| 30 | 723 | 7 |
| 60 | 124 | 1 |

TABLE 15

| | | | | | | relative | loss |
|---|---|---|---|---|---|---|---|
| | | Duration | | | Gel layer | gel | in |
| Temperature 1 | Temperature 2 | stage 1 | Solubility | DP2 | thickness | layer | DS |
| [° C.] | [° C.] | [h] | [%] | [%] | [%] | [%] | [%] |
| 60 | 60 | 3 | 85.8 | 75.5 | 25.4 | | |
| 55 | 60 | 3 | 80.6 | 71.8 | 13.7 | 100 | 8.3 |
| 50 | 60 | 3 | 77.5 | 71.1 | 6.9 | 54 | 4.5 |

Experimental Paramenters

| | | |
|---|---|---|
| 2 | AAU/gds | Spezyme |
| | | Xtra |
| 2 | ASPU/gds | Optimax L- |
| | | 1000 |
| 2.52 | DP/gds | Optimalt |
| | | BBA |

Not wishing to be bound by the theory, this finding may be explained by the fact that two different phenomena are being separated by the temperature staging: 1) the swelling and 2) the gelatinization of the starch. It may be that when the starch granules could swell at a temperature below the gelatinization temperature, amylose may leak out of the granules and may be hydrolyzed before the actual gelatinization event starts. There may be ‘lesser granule’ left to gelatinize and therefore less water may be taken up into the granule, leading to less physical hindrance between the granules, resulting in a lower viscosity peak. What also may play a role is annealing. Annealing may be active between the glass transition temperature and the onset temperature of gelatinization and may result in an improved crystallinity of the granules, which then results in less swelling power of the granules.

### Effect of Temperature Staging on Gel-Formation

Another parameter to analyze the problems relating to the gel formation during malto-saccarification is by performing a filtration test on the resulting fermentation product. For filtration experiments, 1400 g end-of-saccharification slurry is filtered with a Büchner funnel (ø 24 cm) and a vacuum pump into an Erlenmeyer flask with volume indication per 100 mL. As can be seen in Table 16, incubation of fermentation reaction mixture at 60° C. results in a very slow filtration rate. However, incubating the fermentation reaction at 50° C. for a suitable time leads to reduction in filtration time.

TABLE 16

| | | Duration | | | Gel layer | Filtration speed |
|---|---|---|---|---|---|---|
| Temperature 1 | Temperature 2 | stage 1 | Solubility | DP2 | thickness | [% after 3 min of |
| [° C.] | [° C.] | [h] | [%] | [%] | [%] | filtration] |
| 60 | 60 | — | 81.3 | 71.4 | 18.2 | 27 |
| 50 | 60 | 1 | 77.4 | 72.3 | 11.6 | 52 |
| 50 | 60 | 2 | 76.0 | 72.4 | 7.6 | 56 |
| 50 | 60 | 3 | 74.6 | 73.0 | 9.3 | 73 |
| | | | | | | 100* |

Parameters:

| | | |
|---|---|---|
| 2 | AAU/gds | Spezyme |
| | | Xtra |
| 2 | ASPU/gds | Optimax L- |
| | | 1000 |
| 2.52 | DP/gds | Optimalt |
| | | BBA |

1. A method of making a high DP2 syrup containing at least 50% DP2 comprising;
solubilizing a granular starch substrate at or below the initial gelatinization temperature with an exogenous alpha-amylase to form a mixture comprising dextrins;
hydrolyzing the dextrins with a maltogenic enzyme to form the high DP2 syrup, wherein the ratio of alpha-amylase dose expressed as AAU/gds, to maltogenic enzyme dose expressed as DP degrees, is less than 8.

2-3. (canceled)

4. The method according to claim 1 further comprising a debranching enzyme

5. (canceled)

6. The method according to claim 1 wherein the starch is whole ground grain selected from the group consisting of corn, wheat, barley, rye, triticale, rice, oat, beans, banana, potato, sweet potato, sorghum, legumes, cassava, millet, potato, or tapioca.

7-12. (canceled)

13. The method according to claim 1 wherein the maltogenic enzyme is selected from the group consisting of a barley beta-amylase OPTIMALT® BBA or Betalase 1500, a wheat beta-amylase, a soy beta-amylase β-amylase#1500S, a fungal alpha-amylase CLARASE® L, maltogenic amylase is MAX-LIFE™ P100 or Maltogenase L.

14-19. (canceled)

20. The method according to claim 1 wherein the maltogenic enzyme is Fungamyl 800L, Novamyl (Bacillus sp.), GC 626 (AKAA), Veron® XTENDER, beta-amylases from plant material (pea, sweet potato, rye, oats, rice, sorghum), a microbial beta-amylase (paenibacillus polymyxa, Thermoanaerobacterium thermosulfurigenes, Xanthophyllomyces dendrorhous, Bacillus cereus, Bacillus megaterium, Arabidopsis thaliana), a fungal alpha-amylase from Aspergillus (e.g., A. Niger, A. kawachi, and A. oryzae), Trichoderma sp. (e.g. Trichoderma reesei alpha-amylase), Rhisopus sp., Mucor sp., and Penicillium sp., or a maltogenic amylase from Bacillus sp., Bacillus subtilis, Geobacillus stearothermophilus, Lactobacillus gasseri and Thermus sp.

21. The method according to claim 1 wherein the alpha-amylase is selected from the group consisting of SPEZYME® XTRA, SPEZYME® FRED, SPEZYME® LT 300, BAN® 480L, Liquozyme® Supra, Liquozyme® SCDS, MAX-LIFE™ P100, Maltogenase L, CLARASE® L, Liquozyme® SC, Termamyl® SC, Fuelzyme® LF, Veretase, Liqozyme® SC4x, Liquozyme® Supra 2.8, Liquozyme® supra 2, Liquozyme® X, Termamyl® 120L, SPEZYME® ALPHA, SPEZYME® CL, Clearflow® AA, Optitherm™ and Takatherm™, Keistase™, an alpha-amylase from Bacillus species including B. subtilis, B. stearothermophilus, B. lentus, B. licheniformis, B. coagulans, and B. amyloliquefaciens, fungal alpha-amylases from Aspergillus (e.g., A. Niger, A. kawachi, and A. oryzae), Trichoderma sp. (e.g. Trichoderma reesei alpha-amylase), Rhisopus sp., Mucor sp., and Penicillium sp., or a maltogenic amylases from Bacillus sp., Bacillus subtilis, Geobacillus stearothermophilus, Lactobacillus gasseri and Thermus sp.

22. (canceled)

23. The method according to claim 1 wherein the debranching enzyme, if present, is selected from the group consisting of OPTIMAX® L-1000, Promozyme® D2, or ISOAMYLASE from Pseudomonas sp. (e.g. Megazyme), Promozyme D6 (Novozymes), pullulanases secreted by a Bacillus species such as Bacillus deramificans, Bacillus acidopullulyticus, and Bacillus naganoensis, pullulanase from Sulfolobus solfataricus, Pseudomonas sp. and thermostable pullulanase from Fervidobacterium nodosum.

24. (canceled)

25. A composition comprising an alpha-amylase and a maltogenic enzyme wherein the ratio of alpha-amylase dose expressed as AAU/gds, to maltogenic enzyme dose expressed as DP degrees, is 0.002-7.94.

26. (canceled)

27. The composition according to claim 25 further comprising a pullulanase.

28. The composition according to claim 25 further comprising granular starch.

29-31. (canceled)

32. A method of making high DP2 syrup containing at least 50% DP2 comprising; solubilizing a granular starch substrate below the initial gelatinization temperature with an exogenous alpha-amylase and exogenous beta amylase; and
hydrolyzing the resulting mixture at a higher temperature than the first temperature, both of which are below the initial gelatinization temperature, to form the high DP2 syrup.

33. A method according to claim 32 wherein the first temperature is kept for 1 to 3 hours.

34. A method according to claim 32 wherein the first temperature is about 50° C.

35. A method according to claim 32 wherein the second temperature is about 55° C. to about 60° C.

36-39. (canceled)

40. A method according to claim 32 wherein the peak viscosity is reduced by at least about 10% compared to the process when done at the higher temperature only.

41. A method according to claim 32 wherein the filtration speed is improved by at least about 1.5-fold compared to the process when done at the higher temperature only.

42-43. (canceled)
